(19) United States
(12) Patent Application Publication
(10) Pub. No.: US 2018/0280319 A1
(43) Pub. Date: Oct. 4, 2018

(54) COMPOUNDS AND COMPOSITIONS FOR TREATMENT OF BREAST CANCER

(71) Applicant: UNIVERSITY OF THE SCIENCES, PHILADELPHIA, PA (US)

(72) Inventors: ADEBOYE ADEJARE, MANTUA, NJ (US); ISABELLE MERCIER, PHILADELPHIA, PA (US); ZEYNEP ATES-ALAGOZ, ANKARA (TR)

(21) Appl. No.: 15/945,073

(22) Filed: Apr. 4, 2018

Related U.S. Application Data
(60) Provisional application No. 62/481,192, filed on Apr. 4, 2017.

(51) Int. Cl. A61K 31/10; A61P 35/00; A61K 45/06

(52) U.S. Cl. CPC A61K 31/10; A61K 45/06; A61P 35/00

(57) ABSTRACT

The present invention provides novel methods of treating triple-negative breast cancer (TNBC). In certain embodiments, the methods of the invention do not require the use of ionizing radiation therapies. In other embodiments, the methods of the invention do not harm non-cancerous cells.

### CROSS-REFERENCE TO RELATED APPLICATIONS

This application claims priority under 35 U.S.C. §119(e) to U.S. Provisional Application No. 62/481,192, filed Apr. 4, 2017, which is incorporated herein by reference in its entirety.

### BACKGROUND OF THE INVENTION

Triple negative breast cancers (TNBC) are a subset of breast cancers that are characterized by lack of immunohistochemical expression of estrogen receptor (ER), progesterone receptor (PR) and human epidermal growth factor receptor 2 (Her2/neu). TNBCs account for 15-20% of all breast cancers, and are by far the most aggressive form of breast cancer with the poorest prognosis. TNBCs exhibit the shortest overall survival, and characteristically reveal a premature peak of local and distant recurrences (metastases) about three years following the initial diagnosis. These distant relapses exist as visceral and/or brain metastases, and contribute to the aggressive clinical features of TNBC. Unlike receptor-positive cancers, current methods of treatment for TNBCs known in the art are limited to surgery, radiation therapy and highly toxic systemic chemotherapy, because there is an overall lack of targeted therapy for this cancer subtype.

There remains a need in the art for compounds and methods that can be used to treat TNBC in a subject. Further, there is a need in the art for methods of treating TNBC that do not require use of radiation therapies or highly toxic therapies. Such methods should target cancerous cells selectively without harming non-cancerous cells. The present invention meets this need.

### BRIEF SUMMARY OF THE INVENTION

In one aspect, the invention provides a method of treating or preventing triple negative breast cancer (TNBC) in a subject. In certain embodiments, the method comprises administering to the subject a therapeutically effective amount of the compound of formula (I), or a pharmaceutically acceptable salt or solvate thereof:

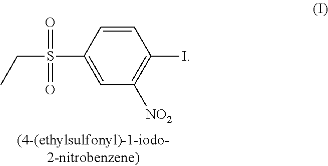

In certain embodiments, the administration of the compound of formula (I) causes apoptosis in TNBC cells. In other embodiments, the administration of the compound of formula (I) does not cause any, or causes insignificant, apoptosis in non-cancerous (normal) cells. In yet other embodiments, the administration of the compound of formula (I) does not cause any, or causes insignificant, apoptosis in non-cancerous cells.

In certain embodiments, the administration of the compound of formula (I) increases the generation of reactive oxygen species in TNBC cells. In other embodiments, the administration of the compound of formula (I) does not increase the generation of reactive oxygen species in non-cancerous cells. In certain embodiments, the administration of the compound of formula (I) induces DNA damage in TNBC cells. In other embodiments, the administration of the compound of formula (I) does not induce DNA damage in non-cancerous cells.

In certain embodiments, the subject is not administered any additional chemotherapeutic agent or anti-cell proliferation agent. In other embodiments, the subject is not administered any additional chemotherapeutic agent or anti-cell proliferation agent in an amount sufficient to treat or prevent TNBC in the subject.

In certain embodiments, the subject is not subjected to ionizing radiation.

In certain embodiments, the method further comprises administering to the subject at least one additional compound selected from the group consisting of a chemotherapeutic agent, an anti-cell proliferation agent, a gene therapy agent, and a immunotherapy agent. In other embodiments, the compound of formula (I) and the at least one additional compound are co-administered to the subject. In yet other embodiments, the compound of formula (I) and the at least one additional compound are coformulated. In yet other embodiments, the at least one additional compound is selected from the group consisting of taxotere, cyclophosphamide, paclitaxel, fluorouracil, doxorubicin, and cycloheximide.

In certain embodiments, the subject is a mammal. In other embodiments, the subject is a human.

### BRIEF DESCRIPTION OF THE DRAWINGS

The following detailed description of specific embodiments of the invention will be better understood when read in conjunction with the appended drawings. For the purpose of illustrating the invention, specific embodiments are shown in the drawings. It should be understood, however, that the invention is not limited to the precise arrangements and instrumentalities of the embodiments shown in the drawings.

FIG. 1A is a set of images illustrating colonies of MDA-MB-231 cells exposed to DMSO (control) and compound 3D (10 μM, 4 hrs). The images show that compound 3D inhibits colony numbers, even in the absence of radiation.

FIG. 1B reports the results depicted in FIG. 1A as a bar graph. These results demonstrate that there are no radiosensitizing effects of compound 3D, as assessed by colony counting; both in the presence and absence of radiation, the % difference between the control and compound 3D was about 40%, regardless of treatment with radiation.

FIG. 2A is a set of chemical structures of parent radiosensitizer NS-123 (4-bromo-3-nitrophenyl propan-1-one) and compound 3D (4-(ethylsulfonyl)-1-iodo-2-nitrobenzene).

FIGS. 2B-2G are images of MDA-MB-231 cells treated with DMSO (control), NS-123 and 3D (FIGS. 2B, 2D and 2F) under a phase contrast microscope (10×) and apoptosis profiles for the respective images (FIGS. 2C, 2E and 2G) determined using flow cytometry. The cells treated with 3D (FIG. 2F) were noticeably misshapen and the 3D annexin V profile showed the appearance of an apoptotic population as seen in the upper right quadrant.

FIG. 2H is a graph showing the results of a quantitative annexin V death assay, quantifying the amount of apoptosis induced by compound 3D.

FIG. 2I is a graph showing the percentage of live TNBC cells after exposure to compound 3D.

FIGS. 3A-3C are a set of apoptotic profiles illustrating caspase 3/7 activation. FIG. 3B illustrates high rates of apoptosis when MDA-MB-231 cells are treated with compound 3D, while FIG. 3A (control) and 3C (4-(ethylsulfonyl)-1-fluoro-2-nitrobenzene) illustrate very low rates of apoptosis.

FIG. 3D is a graph showing percentage of caspase 3/7 positive MDA-MB-231 cells after exposure to control, 3C (4-(ethylsulfonyl)-1-fluoro-2-nitrobenzene), 3B 4-(ethylsulfonyl)-1-chloro-2-nitrobenzene, 3E 4-(ethylsulfonyl)-1-bromo-2-nitrobenzene, 3D (4-(ethylsulfonyl)-1-iodo-2-nitrobenzene) and 1-iodo-2-nitrobenzene (lacking the sulfonyl group, as compared to 3D).

FIG. 4A is a set of images taken of normal MCF-10A mammary cells following treatment with 3D (10 μM, 4 hrs) as well as a graph reporting the percentage of living, apoptotic and necrotic cells after treatment in comparison to control, as reported by an annexin V assay. FIG. 4A demonstrates the fact that 3D has no observable effect on the health of non-TNBC cells.

FIG. 4B is a set of images of MDA-MB-231 (TNBC cells) co-cultured with MCF-10A (normal mammary cells) after exposure to DMSO control (left) as well as 3D (right). FIGS. 5A-5E illustrate the finding that 3D induces a rise in reactive oxygen species (ROS) inside TNBC cells and not normal healthy cells. FIGS. 5A-5C: Following a 4-hour treatment with 3D (10 μM) in MDA-MB-231 cells (TNBC cells), a significant accumulation in a ROS-positive population (red or darker peak) appeared (FIG. 5B, top) when compared to DMSO (vehicle control; FIG. 5A, top) and to the inactive iodo-nitro compound (FIG. 5C, top). When compared to the same treatment in normal MCF-10A cells, 3D compound (10 μM, 4 hrs) was unable to induce a similar ROS buildup as seen in FIG. 5B, bottom, by the absence of the red peak. FIGS. 5D-5E: Graphical representation of ROS accumulation in TNBC cells (FIG. 5D) shows a significant ˜3-fold increase in ROS accumulation following 3D (10 μM, 4 hrs) treatment compared to DMSO p=0.00005, while in normal cells (FIG. 5E) no significant increase was observed at the same dose and time, p=0.9. These results suggest a selective accumulation of ROS inside TNBC cells and not in normal cells following administration of 3D.

FIG. 6A illustrates induction in the phosphorylated levels of ATM (P-ATM ser1981; upper panel) and FIG. 6B illustrates an induction in the phosphorylated levels of H2AX (P-H2AX ser139; lower panel) following a 4 hr-treatment with 3D compound (10 Both ATM and H2AX are involved in DNA damage responses. Phosphorylated levels are reported to either total ATM which remains unchanged or to total H2AX, which also increased slightly suggesting that 3D is driving the total levels of H2AX to be upregulated as well. All results are also reported to a house keeping protein (GAPDH) for loading control. The proteins were assessed by Western Blot analysis by loading 50 μg of protein and running a denaturing SDS page gel. Effects of 3D on DNA damage in MDA-MB-231 cells was compared to a matching dose of DMSO vehicle.

FIGS. 7A-7H illustrate the finding that similar death-inducing effects are observed in other triple negative cells lines BT-549 and Hs578T. As seen in BT-549 TNBC cell line, a 4-hr treatment with 3D compound (10 μM; FIG. 7B) also induced an activated caspase-3/7 population (upper right quadrant) when compared to DMSO (FIG. 7A) and the inactive compound iodo-nitro (FIG. 7C). FIG. 7D represents a graphical quantification of caspase3/7 activation by 3D in BT-549. A similar trend is also observed in HS-578T, another TNBC cell line, which exhibits a similar shift in apoptotic population as seen in 3D-treated cells (4 hrs, 10 μM; FIG. 7F) which shifts to the upper right quadrant compared to DMSO (FIG. 7E) and inactive nitro-iodo compound (FIG. 7G). FIG. 7H represents a graphical quantification of caspase3/7 activation by 3D in HS-578T.

### DETAILED DESCRIPTION OF THE INVENTION

The present invention relates in part to the unexpected discovery that the compound of formula (I), or salts or solvates thereof, can be used to treat triple negative breast cancers (TNBC) in a subject in need thereof:

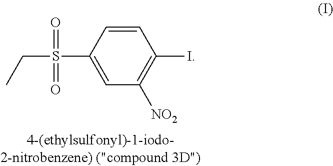

In certain embodiments, the invention provides methods of treating TNBC in a subject, the method comprising administering a therapeutically effective amount of the compound of formula (I), which is optionally formulated as a pharmaceutical composition.

In certain embodiments, the compound of formula (I) treats TNBC without the need for radiation therapy. In other embodiments, the compound of formula (I) is selective for TNBC cells, inducing preferential cellular death and/or apoptosis in TNBC cells as compared to non-cancerous cells.

### Definitions

As used herein, each of the following terms has the meaning associated with it in this section.

Unless defined otherwise, all technical and scientific terms used herein generally have the same meaning as commonly understood by one of ordinary skill in the art to which this invention belongs. Generally, the nomenclature used herein and the laboratory procedures in cell culture, molecular genetics, oncology and organic chemistry are those well-known and commonly employed in the art.

As used herein, the articles “a” and “an” refer to one or to more than one (i.e., to at least one) of the grammatical object of the article. By way of example, “an element” means one element or more than one element.

As used herein, the term “about” is understood by persons of ordinary skill in the art and varies to some extent on the context in which it is used. As used herein when referring to a measurable value such as an amount, a temporal duration, and the like, the term “about” is meant to encompass variations of ±20% or ±10%, more preferably ±5%, even more preferably ±1%, and still more preferably ±0.1% from the specified value, as such variations are appropriate to perform the disclosed methods.

By the term “applicator,” as the term is used herein, is meant any device including, but not limited to, a hypodermic syringe, a pipette, and the like, for administering the compounds and compositions of the invention.

As used herein, the term “cancer” is defined as disease characterized by the rapid and uncontrolled growth of aberrant cells. Cancer cells can spread locally or through the bloodstream and lymphatic system to other parts of the body. Examples of various cancers include but are not limited to, bone cancer, breast cancer, prostate cancer, ovarian cancer, cervical cancer, skin cancer, pancreatic cancer, colorectal cancer, renal cancer, liver cancer, brain cancer, lymphoma, leukemia, lung cancer and the like.

As used herein, a “disorder” in an animal is a state of health in which the animal is able to maintain homeostasis, but in which the animal's state of health is less favorable than it would be in the absence of the disorder. Left untreated, a disorder does not necessarily cause a further decrease in the animal's state of health.

As used herein, the terms “effective amount” or “therapeutically effective amount” or “pharmaceutically effective amount” of a compound are used interchangeably to refer to the amount of the compound that is sufficient to provide a beneficial effect to the subject to which the compound is administered.

As used herein, an “instructional material” includes a publication, a recording, a diagram, or any other medium of expression, which can be used to communicate the usefulness of the compound and/or composition of the invention in the kit for treating or preventing diseases or disorders recited herein. Optionally, or alternately, the instructional material may describe one or more methods of treating or preventing diseases or disorders in a cell or a tissue of a mammal. The instructional material of the kit of the invention may, for example, be affixed to a container, which contains the chemical compound and/or composition of the invention or be shipped together with a container, which contains the chemical composition and/or composition. Alternatively, the instructional material may be shipped separately from the container with the intention that the instructional material and the compound be used cooperatively by the recipient.

A “lesion” is an injury, wound or an area that is structurally abnormal. In the context of a subject bearing tumor, a lesion is a tumor mass unless otherwise described.

A “neoplastic cell,” “cancer cell,” “tumor cell,” or “cell with a proliferative disorder,” refers to a cell that proliferates at an abnormally high rate. A new growth comprising neoplastic cells is a neoplasm, also known as a “tumor.” A tumor is an abnormal tissue growth, generally forming a distinct mass, that grows by cellular proliferation more rapidly than normal tissue growth. A tumor may show partial or total lack of structural organization and functional coordination with normal tissue. As used herein, a tumor is intended to encompass hematopoietic tumors as well as solid tumors.

A tumor may be benign (benign tumor) or malignant (malignant tumor or cancer). Malignant tumors can be broadly classified into three major types. Malignant tumors arising from epithelial structures are called carcinomas, malignant tumors that originate from connective tissues such as muscle, cartilage, fat or bone are called sarcomas and malignant tumors affecting hematopoietic structures (structures pertaining to the formation of blood cells) including components of the immune system, are called leukemias and lymphomas. Other tumors include, but are not limited to neurofibromatosis.

As used herein, the term “pharmaceutically acceptable” refers to a material, such as a carrier or diluent, which does not abrogate the biological activity or properties of the compound, and is relatively non-toxic, i.e., the material may be administered to an individual without causing undesirable biological effects or interacting in a deleterious manner with any of the components of the composition in which it is contained.

The language “pharmaceutically acceptable carrier” includes a pharmaceutically acceptable salt, pharmaceutically acceptable material, composition or carrier, such as a liquid or solid filler, diluent, excipient, solvent or encapsulating material, involved in carrying or transporting a compound(s) of the present invention within or to the subject such that it may perform its intended function. Typically, such compounds are carried or transported from one organ, or portion of the body, to another organ, or portion of the body. Each salt or carrier must be “acceptable” in the sense of being compatible with the other ingredients of the formulation, and not injurious to the subject. Some examples of materials that may serve as pharmaceutically acceptable carriers include: sugars, such as lactose, glucose and sucrose; starches, such as corn starch and potato starch; cellulose, and its derivatives, such as sodium carboxymethyl cellulose, ethyl cellulose and cellulose acetate; powdered tragacanth; malt; gelatin; talc; excipients, such as cocoa butter and suppository waxes; oils, such as peanut oil, cottonseed oil, safflower oil, sesame oil, olive oil, corn oil and soybean oil; glycols, such as propylene glycol; polyols, such as glycerin, sorbitol, mannitol and polyethylene glycol; esters, such as ethyl oleate and ethyl laurate; agar; buffering agents, such as magnesium hydroxide and aluminum hydroxide; alginic acid; pyrogen-free water; isotonic saline; Ringer's solution; ethyl alcohol; phosphate buffer solutions; diluent; granulating agent; lubricant; binder; disintegrating agent; wetting agent; emulsifier; coloring agent; release agent; coating agent; sweetening agent; flavoring agent; perfuming agent; preservative; antioxidant; plasticizer; gelling agent; thickener; hardener; setting agent; suspending agent; surfactant; humectant; carrier; stabilizer; and other non-toxic compatible substances employed in pharmaceutical formulations, or any combination thereof. As used herein, “pharmaceutically acceptable carrier” also includes any and all coatings, antibacterial and antifungal agents, and absorption delaying agents, and the like that are compatible with the activity of the compound, and are physiologically acceptable to the subject. Supplementary active compounds may also be incorporated into the compositions.

As used herein, the language “pharmaceutically acceptable salt” refers to a salt of the administered compounds prepared from pharmaceutically acceptable non-toxic acids, including inorganic acids, organic acids, solvates, hydrates, or clathrates thereof.

As used herein, the term “pharmaceutical composition” refers to a mixture of at least one compound useful within the invention with other chemical components, such as carriers, stabilizers, diluents, dispersing agents, suspending agents, thickening agents, and/or excipients. The pharmaceutical composition facilitates administration of the compound to an organism. Multiple techniques of administering a compound include, but are not limited to, intravenous, oral, aerosol, parenteral, ophthalmic, pulmonary and topical administration.

As used herein, the term “prevent” or “prevention” means no disorder or disease development if none had occurred, or no further disorder or disease development if there had already been development of the disorder or disease. Also considered is the ability of one to prevent some or all of the symptoms associated with the disorder or disease. Disease and disorder are used interchangeably herein.

As used herein, the term “prodrug” refers to a pharmacological substance, drug, formulation or compound that is administered to a subject in an inactive or less active form. Once administered, the prodrug is metabolized in vivo into an active metabolite. A prodrug must undergo chemical or enzymatic conversion by metabolic processes before becoming an active pharmacological agent.

A “prophylactic” treatment is a treatment administered to a subject who does not exhibit signs of a disease or exhibits only early signs of the disease for the purpose of decreasing the risk of developing pathology associated with the disease.

The phrase “reduction of growth,” as used herein, refers to any reduced growth, replication rate, or colony formation exhibited by a neoplastic cell, a cancer cell, or a tumor in response to some therapeutic agent, treatment, or clinical intervention, such as radiation. For example, a neoplastic cell may exhibit a reduction in the cell's growth rate or its ability to replicate and form colonies in vitro or in vivo (e.g., when implanted as a tumor in an animal) in response to radiation.

The phrase “reduction in viability,” as used herein, refers to any reduction in survival exhibited by a neoplastic cell, a cancer cell, or a tumor in response to some chemotherapeutic agent, treatment, or clinical intervention, such as radiation. A neoplastic cell, a cancer cell, or a tumor may exhibit reduced viability in response to any such intervention by inhibition of progression of the cell through the cell cycle; damaged nucleic acids, proteins, or other macromolecules in a cell, induced terminal differentiation (senescence), in which the cell no longer replicates; inhibited cellular repair of nucleic acids; or increased rates of cell death by inducing apoptosis or “mitotic catastrophe”—a form of necrosis, when DNA damage levels are beyond those that can be effectively repaired.

A “therapeutic” treatment is a treatment administered to a subject who exhibits signs of pathology for the purpose of diminishing or eliminating those signs.

“Treating,” as used herein, means reducing the frequency with which symptoms are experienced by a patient or subject, or administering an agent or compound to reduce the severity with which symptoms are experienced by a patient or subject. An appropriate therapeutic amount in any individual case may be determined by one of ordinary skill in the art using routine experimentation.

The term “solvate,” as used herein, refers to a compound formed by solvation.

The term “solvation,” as used herein refers to the process of attraction and association of molecules of a solvent with molecules or ions of a solute. As molecules or ions of a solute dissolve in a solvent, they spread out and become surrounded by solvent molecules.

Throughout this disclosure, various aspects of the invention may be presented in a range format. It should be understood that the description in range format is merely for convenience and brevity and should not be construed as an inflexible limitation on the scope of the invention. Accordingly, the description of a range should be considered to have specifically disclosed all the possible sub-ranges as well as individual numerical values within that range and, when appropriate, partial integers of the numerical values within ranges. For example, description of a range such as from 1 to 6 should be considered to have specifically disclosed sub-ranges such as from 1 to 3, from 1 to 4, from 1 to 5, from 2 to 4, from 2 to 6, from 3 to 6 etc., as well as individual numbers within that range, for example, 1, 2, 2.7, 3, 4, 5, 5.3, and 6. This applies regardless of the breadth of the range.

The following abbreviations are used herein: ER, estrogen receptor; HER2, human epidermal growth factor receptor 2; PR, progesterone receptor; TNBC, Triple negative breast cancer.

### Compositions

In certain embodiments, the invention provides the compound of formula (I), or a pharmaceutically acceptable analogue, derivative, adduct, salt, conjugate, prodrug or solvate thereof:

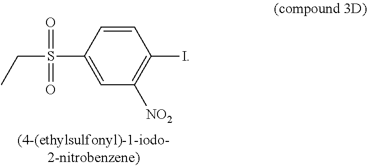

In certain embodiments, the compound of formula (I) induces apoptosis in TNBC cells. In other embodiments, the compound of formula (I) induces apoptosis in TNBC cells preferentially over non-cancerous cells. In yet other embodiments, the compound of formula (I) induces apoptosis in TNBC cells and have no effect, or insignificant effect, on “normal” mammary cell lines (for example, MCF-10A).

### Methods

The present invention provides methods of treating TNBC in a subject in need thereof. In certain embodiments, the method comprises administering to the subject a therapeutically effective amount of the compound of formula (I), or a pharmaceutically acceptable analogue, derivative, conjugate, adduct, salt, prodrug or solvate thereof.

In certain embodiments, the methods of the invention trigger apoptosis in TNBC cells preferentially over non-cancerous cells. In other embodiments, the methods of the invention do not comprise use of ionizing radiation.

In certain embodiments, the composition is formulated as part of an extended-release formulation. In other embodiments, the composition is administered to the subject by at least one route selected from the group consisting of inhalational, oral, rectal, vaginal, parenteral, topical, transdermal, pulmonary, intranasal, buccal, sublingual, ophthalmic, intrathecal, intravenous, and intragastrical.

In certain embodiments, the methods of the invention further comprise administering one or more additional therapeutic agents that treat TNBC. In other embodiments, the methods of the invention further comprise administering one or more therapeutic agents selected from the group consisting of chemotherapeutic agents, anti-cell proliferation agents, gene therapy agents, and immunotherapy agents.

In certain embodiments, the subject is a mammal. In other embodiments, the subject is a human.

In certain embodiments, administering the compound of formula (I) increases the generation of reactive oxygen species in TNBC cells. In other embodiments, administering the compound of formula (I) does not increase generation of reactive oxygen species in non-tumorigenic cells.

In certain embodiments administering the compound of formula (I) induces DNA damage in TNBC cells. In other embodiments, administering the compound of formula (I) does not induce DNA damage in non-tumorigenic cells.

### Combination Therapies

In certain embodiments, the compounds of the present invention are useful in the methods of present invention in combination with one or more additional compounds useful for treating the diseases or disorders contemplated within the invention. These additional compounds may comprise compounds of the present invention or compounds, e.g., commercially available compounds, known to treat, prevent, or reduce the symptoms of the diseases or disorders contemplated within the invention.

Non-limiting examples of additional compounds contemplated within the invention include chemotherapeutic agents, anti-cell proliferation agents, gene therapy agents, and immunotherapy agents. In certain embodiments, the method of the invention can be used in combination with one or more compounds selected from, but not necessarily limited to, the group consisting of taxotere, cyclophosphamide, paclitaxel, fluorouracil, doxorubicin, and cycloheximide. In other embodiments, the method of the invention can be used in combination with any chemotherapeutic, gene therapy or immunotherapy compound or treatment regimen known in the art. In yet other embodiments, the method of the invention can be used in combination with chemotherapeutic compounds known to treat TNBC.

The methods of the present invention can be used in combination with other treatment regimens, including virostatic and virotoxic agents, antibiotic agents, antifungal agents, anti-inflammatory agents (steroidal and non-steroidal), antidepressants, anxiolytics, pain management agents, (acetaminophen, aspirin, ibuprofen, opiates (including morphine, hydrocodone, codeine, fentanyl, methadone)), steroids (including prednisone and dexamethasone), and antidepressants (including gabapentin, amitriptyline, imipramine, doxepin) antihistamines, antitussives, muscle relaxants, bronchodilators, beta-agonists, anticholinergics, corticosteroids, mast cell stabilizers, leukotriene modifiers, methylxanthines, nucleic acid based therapeutic agents, as well as combination therapies, and the like. The compounds of the present invention may be administered before, during, after, or throughout administration of any therapeutic agents used in the treatment of a subject's disease or disorder.

A synergistic effect may be calculated, for example, using suitable methods such as, for example, the Sigmoid-Eₘₐₓ equation (Holford & Scheiner, 19981, Clin. Pharmacokinet. 6: 429-453), the equation of Loewe additivity (Loewe & Muischnek, 1926, Arch. Exp. Pathol Pharmacol. 114: 313-326) and the median-effect equation (Chou & Talalay, 1984, Adv. Enzyme Regul. 22: 27-55). Each equation referred to above may be applied to experimental data to generate a corresponding graph to aid in assessing the effects of the drug combination. The corresponding graphs associated with the equations referred to above are the concentration-effect curve, isobologram curve and combination index curve, respectively.

### Administration/Dosage/Formulations

The regimen of administration may affect what constitutes an effective amount. The therapeutic formulations may be administered to the patient either prior to or after the onset of a disease or disorder. Further, several divided dosages, as well as staggered dosages may be administered daily or sequentially, or the dose may be continuously infused, or may be a bolus injection. Further, the dosages of the therapeutic formulations may be proportionally increased or decreased as indicated by the exigencies of the therapeutic or prophylactic situation.

Administration of the compositions useful within the present invention to a patient, preferably a mammal, more preferably a human, may be carried out using known procedures, at dosages and for periods of time effective to treat a disease or disorder in the patient. An effective amount of the therapeutic compound necessary to achieve a therapeutic effect may vary according to factors such as the state of the disease or disorder in the patient; the age, sex, and weight of the patient; and the ability of the therapeutic compound to treat a disease or disorder in the patient. Dosage regimens may be adjusted to provide the optimum therapeutic response. For example, several divided doses may be administered daily or the dose may be proportionally reduced as indicated by the exigencies of the therapeutic situation. A non-limiting example of an effective dose range for a therapeutic compound of the present invention is from about 1 and 5,000 mg/kg of body weight/per day. One of ordinary skill in the art is able to study the relevant factors and make the determination regarding the effective amount of the therapeutic compound without undue experimentation.

Actual dosage levels of the active ingredients in the pharmaceutical compositions of this invention may be varied so as to obtain an amount of the active ingredient that is effective to achieve the desired therapeutic response for a particular patient, composition, and mode of administration, without being toxic to the patient.

In particular, the selected dosage level depends upon a variety of factors including the activity of the particular compound employed, the time of administration, the rate of excretion of the compound, the duration of the treatment, other drugs, compounds or materials used in combination with the compound, the age, sex, weight, condition, general health and prior medical history of the patient being treated, and like factors well, known in the medical arts.

A medical doctor, e.g., physician or veterinarian, having ordinary skill in the art may readily determine and prescribe the effective amount of the pharmaceutical composition required. For example, the physician or veterinarian could start doses of the compounds of the present invention employed in the pharmaceutical composition at levels lower than that required in order to achieve the desired therapeutic effect and gradually increase the dosage until the desired effect is achieved.

In particular embodiments, it is advantageous to formulate the compound in dosage unit form for ease of administration and uniformity of dosage. Dosage unit form as used herein refers to physically discrete units suited as unitary dosages for the patients to be treated; each unit containing a predetermined quantity of therapeutic compound calculated to produce the desired therapeutic effect in association with the required pharmaceutical vehicle. The dosage unit forms of the present invention are dictated by and directly dependent on the unique characteristics of the therapeutic compound and the particular therapeutic effect to be achieved, and the limitations inherent in the art of compounding/formulating such a therapeutic compound for the treatment of a disease or disorder in a patient.

In certain embodiments, the compositions useful within the invention are formulated using one or more pharmaceutically acceptable excipients or carriers. In certain embodiments, the pharmaceutical compositions of the present invention comprise a therapeutically effective amount of a compound useful within the invention and a pharmaceutically acceptable carrier.

The carrier may be a solvent or dispersion medium containing, for example, water, ethanol, polyol (for example, glycerol, propylene glycol, and liquid polyethylene glycol, and the like), suitable mixtures thereof, and vegetable oils. The proper fluidity may be maintained, for example, by the use of a coating such as lecithin, by the maintenance of the required particle size in the case of dispersion and by the use of surfactants. Prevention of the action of microorganisms may be achieved by various antibacterial and antifungal agents, for example, parabens, chlorobutanol, phenol, ascorbic acid, thimerosal, and the like. In many cases, it is preferable to include isotonic agents, for example, sugars, sodium chloride, or polyalcohols such as mannitol and sorbitol, in the composition. Prolonged absorption of the injectable compositions may be brought about by including in the composition an agent which delays absorption, for example, aluminum monostearate or gelatin.

In certain embodiments, the compositions useful within the invention are administered to the patient in dosages that range from one to five times per day or more. In other embodiments, the compositions useful within the invention are administered to the patient in range of dosages that include, but are not limited to, once every day, every two, days, every three days to once a week, and once every two weeks. It is readily apparent to one skilled in the art that the frequency of administration of the various combination compositions useful within the invention varies from individual to individual depending on many factors including, but not limited to, age, disease or disorder to be treated, gender, overall health, and other factors. Thus, the invention should not be construed to be limited to any particular dosage regime and the precise dosage and composition to be administered to any patient is determined by the attending physician taking all other factors about the patient into account.

Compounds for administration may be in the range of from about 1 μg to about 10,000 mg, about 20 μg to about 9,500 mg, about 40 μg to about 9,000 mg, about 75 μg to about 8,500 mg, about 150 μg to about 7,500 mg, about 200 μg to about 7,000 mg, about 3050 μg to about 6,000 mg, about 500 μg to about 5,000 mg, about 750 μg to about 4,000 mg, about 1 mg to about 3,000 mg, about 10 mg to about 2,500 mg, about 20 mg to about 2,000 mg, about 25 mg to about 1,500 mg, about 50 mg to about 1,000 mg, about 75 mg to about 900 mg, about 100 mg to about 800 mg, about 250 mg to about 750 mg, about 300 mg to about 600 mg, about 400 mg to about 500 mg, and any and all whole or partial increments therebetween.

In some embodiments, the dose of a compound is from about 1 mg and about 2,500 mg. In some embodiments, a dose of a compound of the present invention used in compositions described herein is less than about 10,000 mg, or less than about 8,000 mg, or less than about 6,000 mg, or less than about 5,000 mg, or less than about 3,000 mg, or less than about 2,000 mg, or less than about 1,000 mg, or less than about 500 mg, or less than about 200 mg, or less than about 50 mg. Similarly, in some embodiments, a dose of a second compound (i.e., a drug used for treating a disease or disorder) as described herein is less than about 1,000 mg, or less than about 800 mg, or less than about 600 mg, or less than about 500 mg, or less than about 400 mg, or less than about 300 mg, or less than about 200 mg, or less than about 100 mg, or less than about 50 mg, or less than about 40 mg, or less than about 30 mg, or less than about 25 mg, or less than about 20 mg, or less than about 15 mg, or less than about 10 mg, or less than about 5 mg, or less than about 2 mg, or less than about 1 mg, or less than about 0.5 mg, and any and all whole or partial increments thereof.

In certain embodiments, the drug is therapeutically active at a circulating and/or tissue concentration of about 1, 2, 4, 6, 8, 10, 12, 14, 16, 18, 20, 25, 30, 35, 40, 45 or 50 μM.

In certain embodiments, the present invention is directed to a packaged pharmaceutical composition comprising a container holding a therapeutically effective amount of a compound of the present invention, alone or in combination with a second pharmaceutical agent; and instructions for using the compound to treat, prevent, or reduce one or more symptoms of a disease or disorder in a patient.

Formulations may be employed in admixtures with conventional excipients, i.e., pharmaceutically acceptable organic or inorganic carrier substances suitable for oral, parenteral, nasal, intravenous, subcutaneous, enteral, or any other suitable mode of administration, known to the art. The pharmaceutical preparations may be sterilized and if desired mixed with auxiliary agents, e.g., lubricants, preservatives, stabilizers, wetting agents, emulsifiers, salts for influencing osmotic pressure, buffers, coloring, flavoring and/or aromatic substances and the like. They may also be combined where desired with other active agents, e.g., other anti-tumor agents.

The term “container” includes any receptacle for holding the pharmaceutical composition. For example, in certain embodiments, the container is the packaging that contains the pharmaceutical composition. In other embodiments, the container is not the packaging that contains the pharmaceutical composition, i.e., the container is a receptacle, such as a box or vial that contains the packaged pharmaceutical composition or unpackaged pharmaceutical composition and the instructions for use of the pharmaceutical composition. Moreover, packaging techniques are well known in the art. It should be understood that the instructions for use of the pharmaceutical composition may be contained on the packaging containing the pharmaceutical composition, and as such the instructions form an increased functional relationship to the packaged product. However, it should be understood that the instructions may contain information pertaining to the compound's ability to perform its intended function, e.g., treating, preventing, or reducing a disease or disorder in a patient.

Routes of administration of any of the compositions of the present invention include oral, nasal, rectal, intravaginal, parenteral, buccal, sublingual or topical. The compounds for use in the invention may be formulated for administration by any suitable route, such as for oral or parenteral, for example, transdermal, transmucosal (e.g., sublingual, lingual, (trans)buccal, (trans)urethral, vaginal (e.g., trans- and perivaginally), (intra)nasal and (trans)rectal), intravesical, intrapulmonary, intraduodenal, intragastrical, intrathecal, subcutaneous, intramuscular, intradermal, intra-arterial, intravenous, intrabronchial, inhalation, and topical administration.

Suitable compositions and dosage forms include, for example, tablets, capsules, caplets, pills, gel caps, troches, dispersions, suspensions, solutions, syrups, granules, beads, transdermal patches, gels, powders, pellets, magmas, lozenges, creams, pastes, plasters, lotions, discs, suppositories, liquid sprays for nasal or oral administration, dry powder or aerosolized formulations for inhalation, compositions and formulations for intravesical administration and the like. It should be understood that the formulations and compositions that would be useful in the present invention are not limited to the particular formulations and compositions that are described herein.

### Oral Administration

For oral administration, particularly suitable are tablets, dragees, liquids, drops, or capsules, caplets and gelcaps. The compositions intended for oral use may be prepared according to any method known in the art and such compositions may contain one or more agents selected from the group consisting of inert, non-toxic pharmaceutically acceptable excipients which are suitable for the manufacture of tablets. Such excipients include, for example an inert diluent such as lactose; granulating and disintegrating agents such as cornstarch; binding agents such as starch; and lubricating agents such as magnesium stearate. The tablets may be uncoated or they may be coated by known techniques for elegance or to delay the release of the active ingredients. Formulations for oral use may also be presented as hard gelatin capsules wherein the active ingredient is mixed with an inert diluent.

For oral administration, the compounds may be in the form of tablets or capsules prepared by conventional means with pharmaceutically acceptable excipients such as binding agents (e.g., polyvinylpyrrolidone, hydroxypropylcellulose or hydroxypropylmethylcellulose); fillers (e.g., cornstarch, lactose, microcrystalline cellulose or calcium phosphate); lubricants (e.g., magnesium stearate, talc, or silica); disintegrates (e.g., sodium starch glycollate); or wetting agents (e.g., sodium lauryl sulfate). If desired, the tablets may be coated using suitable methods and coating materials such as OPADRY™ film coating systems available from Colorcon, West Point, Pa. (e.g., OPADRY™ OY Type, OYC Type, Organic Enteric OY-P Type, Aqueous Enteric OY-A Type, OY-PM Type and OPADRY™ White, 32K18400). Liquid preparation for oral administration may be in the form of solutions, syrups or suspensions. The liquid preparations may be prepared by conventional means with pharmaceutically acceptable additives such as suspending agents (e.g., sorbitol syrup, methyl cellulose or hydrogenated edible fats); emulsifying agent (e.g., lecithin or acacia); non-aqueous vehicles (e.g., almond oil, oily esters or ethyl alcohol); and preservatives (e.g., methyl or propyl p-hydroxy benzoates or sorbic acid).

Granulating techniques are well known in the pharmaceutical art for modifying starting powders or other particulate materials of an active ingredient. The powders are typically mixed with a binder material into larger permanent free-flowing agglomerates or granules in a process referred to as “granulation.” For example, solvent-using “wet” granulation processes are generally characterized in that the powders are combined with a binder material and moistened with water or an organic solvent under conditions resulting in the formation of a wet granulated mass from which the solvent must then be evaporated.

Melt granulation generally consists in the use of materials that are solid or semi-solid at room temperature (i.e. having a relatively low softening or melting point range) to promote granulation of powdered or other materials, essentially in the absence of added water or other liquid solvents. The low melting solids, when heated to a temperature in the melting point range, liquefy to act as a binder or granulating medium. The liquefied solid spreads itself over the surface of powdered materials with which it is contacted, and on cooling, forms a solid granulated mass in which the initial materials are bound together. The resulting melt granulation may then be provided to a tablet press or be encapsulated for preparing the oral dosage form. Melt granulation improves the dissolution rate and bioavailability of a drug by forming a solid dispersion or solid solution.

U.S. Pat. No. 5,169,645 discloses directly compressible wax-containing granules having improved flow properties. The granules are obtained when waxes are admixed in the melt with certain flow improving additives, followed by cooling and granulation of the admixture. In certain embodiments, only the wax itself melts in the melt combination of the wax(es) and additives(s), and in other cases both the wax(es) and the additives(s) melt.

The present invention also includes a multi-layer tablet comprising a layer providing for the delayed release of one or more compounds of the present invention, and a further layer providing for the immediate release of a medication for treatment of a disease or disorder. Using a wax/pH-sensitive polymer mix, a gastric insoluble composition may be obtained in which the active ingredient is entrapped, ensuring its delayed release.

### Parenteral Administration

For parenteral administration, the compounds may be formulated for injection or infusion, for example, intravenous, intramuscular or subcutaneous injection or infusion, or for administration in a bolus dose and/or continuous infusion. Suspensions, solutions or emulsions in an oily or aqueous vehicle, optionally containing other formulatory agents such as suspending, stabilizing and/or dispersing agents may be used.

### Additional Administration Forms

Additional dosage forms of this invention include dosage forms as described in U.S. Pat. Nos. 6,340,475, 6,488,962, 6,451,808, 5,972,389, 5,582,837, and 5,007,790. Additional dosage forms of this invention also include dosage forms as described in U.S. Patent Applications Nos. 20030147952, 20030104062, 20030104053, 20030044466, 20030039688, and 20020051820. Additional dosage forms of this invention also include dosage forms as described in PCT Applications Nos. WO 03/35041, WO 03/35040, WO 03/35029, WO 03/35177, WO 03/35039, WO 02/96404, WO 02/32416, WO 01/97783, WO 01/56544, WO 01/32217, WO 98/55107, WO 98/11879, WO 97/47285, WO 93/18755, and WO 90/11757.

### Controlled Release Formulations and Drug Delivery Systems

In certain embodiments, the formulations of the present invention may be, but are not limited to, short-term, rapid-offset, as well as controlled, for example, sustained release, delayed release and pulsatile release formulations.

The term sustained release is used in its conventional sense to refer to a drug formulation that provides for gradual release of a drug over an extended period of time, and that may, although not necessarily, result in substantially constant blood levels of a drug over an extended time period. The period of time may be as long as a month or more and should be a release which is longer that the same amount of agent administered in bolus form.

For sustained release, the compounds may be formulated with a suitable polymer or hydrophobic material which provides sustained release properties to the compounds. As such, the compounds for use the method of the present invention may be administered in the form of microparticles, for example, by injection or in the form of wafers or discs by implantation.

In certain embodiments, the compounds of the present invention are administered to a patient, alone or in combination with another pharmaceutical agent, using a sustained release formulation.

The term delayed release is used herein in its conventional sense to refer to a drug formulation that provides for an initial release of the drug after some delay following drug administration and that may, although not necessarily, include a delay of from about 10 minutes up to about 12 hours.

The term pulsatile release is used herein in its conventional sense to refer to a drug formulation that provides release of the drug in such a way as to produce pulsed plasma profiles of the drug after drug administration.

The term immediate release is used in its conventional sense to refer to a drug formulation that provides for release of the drug immediately after drug administration.

As used herein, short-term refers to any period of time up to and including about 8 hours, about 7 hours, about 6 hours, about 5 hours, about 4 hours, about 3 hours, about 2 hours, about 1 hour, about 40 minutes, about 20 minutes, or about 10 minutes and any or all whole or partial increments thereof after drug administration.

As used herein, rapid-offset refers to any period of time up to and including about 8 hours, about 7 hours, about 6 hours, about 5 hours, about 4 hours, about 3 hours, about 2 hours, about 1 hour, about 40 minutes, about 20 minutes, or about 10 minutes, and any and all whole or partial increments thereof after drug administration.

### Dosing

The therapeutically effective amount or dose of a compound depends on the age, sex and weight of the patient, the current medical condition of the patient and the progression of the disease or disorder in the patient being treated. The skilled artisan is able to determine appropriate dosages depending on these and other factors.

A suitable dose of a compound of the present invention may be in the range of from about 0.01 mg to about 5,000 mg per day, such as from about 0.1 mg to about 1,000 mg, for example, from about 1 mg to about 500 mg, such as about 5 mg to about 250 mg per day. The dose may be administered in a single dosage or in multiple dosages, for example from 1 to 4 or more times per day. When multiple dosages are used, the amount of each dosage may be the same or different. For example, a dose of 1 mg per day may be administered as two 0.5 mg doses, with about a 12-hour interval between doses.

It is understood that the amount of compound dosed per day may be administered, in non-limiting examples, every day, every other day, every 2 days, every 3 days, every 4 days, or every 5 days. For example, with every other day administration, a 5 mg per day dose may be initiated on Monday with a first subsequent 5 mg per day dose administered on Wednesday, a second subsequent 5 mg per day dose administered on Friday, and so on.

The compounds for use in the method of the present invention may be formulated in unit dosage form. The term “unit dosage form” refers to physically discrete units suitable as unitary dosage for patients undergoing treatment, with each unit containing a predetermined quantity of active material calculated to produce the desired therapeutic effect, optionally in association with a suitable pharmaceutical carrier. The unit dosage form may be for a single daily dose or one of multiple daily doses (e.g., about 1 to 4 or more times per day). When multiple daily doses are used, the unit dosage form may be the same or different for each dose.

Those skilled in the art will recognize, or be able to ascertain using no more than routine experimentation, numerous equivalents to the specific procedures, embodiments, claims, and examples described herein. Such equivalents were considered to be within the scope of this invention and covered by the claims appended hereto. For example, it should be understood, that modifications in reaction conditions, including but not limited to reaction times, reaction size/volume, and experimental reagents, such as solvents, catalysts, pressures, atmospheric conditions, e.g., nitrogen atmosphere, and reducing/oxidizing agents, with art-recognized alternatives and using no more than routine experimentation, are within the scope of the present application.

It is to be understood that, wherever values and ranges are provided herein, the description in range format is merely for convenience and brevity and should not be construed as an inflexible limitation on the scope of the invention. Accordingly, all values and ranges encompassed by these values and ranges are meant to be encompassed within the scope of the present invention. Moreover, all values that fall within these ranges, as well as the upper or lower limits of a range of values, are also contemplated by the present application. The description of a range should be considered to have specifically disclosed all the possible sub-ranges as well as individual numerical values within that range and, when appropriate, partial integers of the numerical values within ranges. For example, description of a range such as from 1 to 6 should be considered to have specifically disclosed sub-ranges such as from 1 to 3, from 1 to 4, from 1 to 5, from 2 to 4, from 2 to 6, from 3 to 6 etc., as well as individual numbers within that range, for example, 1, 2, 2.7, 3, 4, 5, 5.3, and 6. This applies regardless of the breadth of the range.

The following examples further illustrate aspects of the present invention. However, they are in no way a limitation of the teachings or disclosure of the present invention as set forth herein.

### EXAMPLES

The invention is now described with reference to the following Examples. These Examples are provided for the purpose of illustration only, and the invention is not limited to these Examples, but rather encompasses all variations that are evident as a result of the teachings provided herein.

### Materials and Methods

### 1-(Ethylsulfonyl)-4-fluorobenzene

A reddish solution of sodium telluride, prepared by heating a mixture of powdered tellurium (10 mmol, 1.28 g), Rongalite (also known as formaldehydedesulfoxylate dihydrate) (50 mmol, 7.71 g) and 1 M aqueous sodium hydroxide (25 ml), was added dropwise to a stirred solution of p-fluoro-sulfonyl chloride (10 mmol, 1.94 g) and triethylbenzyl ammonium chloride (TEBAC) (0.1 mmol, 0.23 g) in THF (30 ml) at room temperature under nitrogen. An instantaneous reaction occurred and the color of the reaction mixture changed to deep black. After 5 minutes while stirring, iodoethane (50 mmol, 4 ml) in THF (3 ml) was added and the resulting mixture was kept at 90°C. for 5 hours. After cooling, the solvent was removed under reduced pressure and the residue was treated with aqueous ammonium chloride and benzene. Organic phase was separated, dried over sodium sulfate, and the solvent evaporated. The residue was purified by column chromatography (hexane/ethylacetate 1:1) to give a white solid which was crystallized from chloroform and hexane. HPLC indicated 94% purity, mp 39-40°C. (0.30 g, 16% yield). ¹H NMR (CDCl₃): 1.6 (t, 3H, CH₃), 3.23 (q, 2H, CH₂), 7.27 (m, 2H, Ar—H), 7.94 (m, 2H, Ar—H); MS: 189 M. Elemental Analysis (C, H, F, S) for C₈H₉FO₂S; calculated C, 51.05; H, 4.82; F, 10.09; S, 17.04. found C, 51.07; H, 4.67; F, 10.36; S, 16.85.

### 1-(Ethylsulfonyl)-4-iodobenzene

A reddish solution of sodium telluride, prepared by heating a mixture of powdered tellurium (10 mmol, 1.28 g), Rongalite (also known as formaldehydedesulfoxylate dihydrate) (50 mmol, 7.71 g) and 1 M aqueous sodium hydroxide (25 ml), was added dropwise to a stirred solution of 4-iodobenzenesulfonyl chloride (10 mmol, 3.02 g) and triethylbenzylammonium chloride (TEBAC) (0.1 mmol, 0.23 g) in THF (30 ml) at room temperature under nitrogen. An instantaneous reaction occurred and the color of the reaction mixture changed to deep black. After 5 minutes while stirring, iodoethane (50 mmol, 4 ml) in THF (3 ml) was added and the resulting mixture was kept at 90°C. for 5 hours. After cooling, the solvent was removed under reduced pressure and the residue was treated with aqueous ammonium chloride and benzene. Organic phase was separated, dried over sodium sulfate, and the solvent evaporated. The residue was purified by column chromatography (hexane/ethylacetate 1:1). A white solid was crystallized from ethyl acetate and hexane. HPLC indicated 100% purity, mp 78-79°C. (0.60 g, 20% yield). ¹H NMR (CDCl₃): 1.28 (t, 3H, CH₃), 3.12 (q, 2H, CH₂), 7.63 (d, 2H, Ar—H), 7.96 (d, 2H, Ar—H); MS: 296 M. Elemental Analysis (C, H, I, S) for C₈H₉IO₂S.0.2C₆H₁₄-0.1H₂O;

calculated C, 35.06; H, 3.83; I, 40.26; S, 10.17. found C, 35.04; H, 3.41; I, 40.32; S, 9.74.

### 1-(Ethylsulfonyl)-4-chlorobenzene

1-(Ethyl sulfonyl)-4-chlorobenzene was prepared in analogy with 1-(Ethyl sulfonyl)-4-fluorobenzene and 1-(Ethylsulfonyl)-4-iodobenzene, as described elsewhere herein, starting from 4-chlorobenzenesulfonyl chloride. ¹H NMR (400MHz, CDCl₃) δ ppm 7.86 (d, J=8.53Hz, 2H), 7.56 (d, J=8.56Hz, 2H), 3.13 (q, J=7.43Hz, 2H), 1.29 (t, J=7.44Hz, 3H); MS (ESI, +ion): 205 M⁺ (100.0%), 207 (36.5%).

### 1-(Ethylsulfonyl)-4-bromobenzene

1-(Ethyl sulfonyl)-4-bromobenzene was prepared in analogy with 1-(Ethyl sulfonyl)-4-fluorobenzene and 1-(Ethylsulfonyl)-4-iodobenzene, as described above, starting from 4-bromobenzenesulfonyl chloride.

### 4-(Ethylsulfonyl)-1-fluoro-2-nitrobenzene (3C)

To a solution of 1-(ethylsulfonyl)-4-fluorobenzene (1.36 mmol, 0.257 g) in sulfuric acid (1.32 ml) was added potassium nitrate (0.243 g) at 80°C. The mixture was stirred at 90°C. for 2 hours. Ice water (5 ml) was added and the mixture was extracted with ethyl acetate (25 ml) and washed with water (20 ml) and brine (10 ml). The organic extracts were combined and dried over sodium sulfate and concentrated. The residue was purified by column chromatography (hexane/ethylacetate 1:3) to give a light yellow solid which was crystallized from methanol. HPLC indicated 98% purity, mp 128-131°C. (0.21 g, 66% yield). ¹H NMR (CDCl₃): 1.35 (t, 3H, CH₃), 3.21 (q, 2H, CH₂), 7.55 (t, 1H, Ar—H), 8.21 (m, 1H, Ar—H), 8.64 (d, 1H, Ar—H); MS: 229 M⁺. Elemental Analysis (C, H, N, F, S) for C₈H₈FNO₄S; calculated C, 41.20; H, 3.46; N, 6.01; F, 8.15; S, 13.75. found C, 41.31; H, 3.35; N, 5.85; F, 8.17; S, 13.53.

### 4-(Ethylsulfonyl)-1-iodo-2-nitrobenzene (3D)

Compound 3D was prepared in analogy to 4-(Ethylsulfonyl)-1-fluoro-2-nitrobenzene, starting from 1-(ethylsulfonyl)-4-iodobenzene (0.34 mmol, 0.10 g) to give a light yellow solid which was crystallized from ethyl acetate and hexane. HPLC indicated 98% purity, mp 124-126°C. (0.052 g, 45% yield). ¹H NMR (CDCl₃): 1.35 (t, 3H, CH₃), 3.22 (q, 2H, CH₂), 7.77 (dd, 1H, Ar—H), 8.31 (d, 1H, Ar—H), 8.34 (d, 1H, Ar—H); MS: 338.9 M⁻. Elemental Analysis (C, H, N, I, S) for C₈H₈INO₄S; calculated C, 28.17; H, 2.36; N, 4.11; I, 37.20; S, 9.40. found C, 28.25; H, 2.25; N, 3.99; I, 37.39; S, 9.18.

### 4-(Ethylsulfonyl)-1-chloro-2-nitrobenzene

4-(Ethylsulfonyl)-1-chloro-2-nitrobenzene was prepared in analogy with 4-(Ethylsulfonyl)-1-fluoro-2-nitrobenzene, as described above, starting from 1-(Ethylsulfonyl)-4-chlorobenzene. ¹H NMR (400 MHz, CDCl₃) δ ppm 8.42 (d, J=2.03Hz, 1H), 8.06 (dd, 8.39Hz, 1H), 7.83 (t, J=6.45Hz, 1H), 3.21 (q, J=7.44Hz, 2H), 1.36 (t, J=7.44Hz, 3H); MS (ESI, +ion): 249.99, M⁺ (100.00%), 251.00 (36.5). Anal. calcd. for C₈H₈ClNO₄S: C, 38.48; H, 3.23; N, 5.61; Cl, 14.20; S, 12.84 and found C, 38.79; H, 3.23; N, 5.25; Cl, 14.41; S, 12.64.

### 4-(Ethylsulfonyl)-1-bromo-2-nitrobenzene

4-(Ethylsulfonyl)-1-bromo-2-nitrobenzene was prepared in analogy with 4-(Ethylsulfonyl)-1-fluoro-2-nitrobenzene, as described above, starting from 1-(Ethylsulfonyl)-4-bromobenzene.

### 1-iodo-2-nitrobenzene

1-iodo-2-nitrobenzene was purchased from a commercial source, and used as is.

### EXAMPLE 1

### Compound 3D Failed to Radiosensitize TNBC MDA-MB-231 Cells

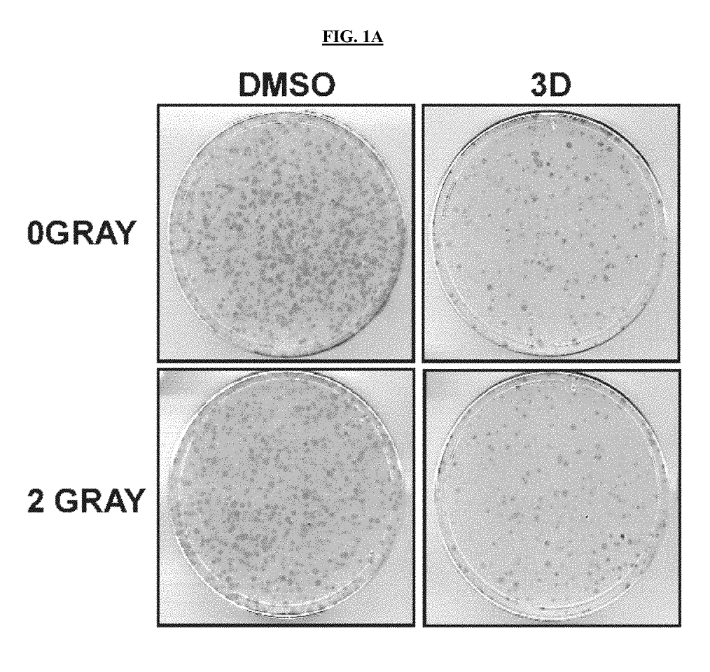

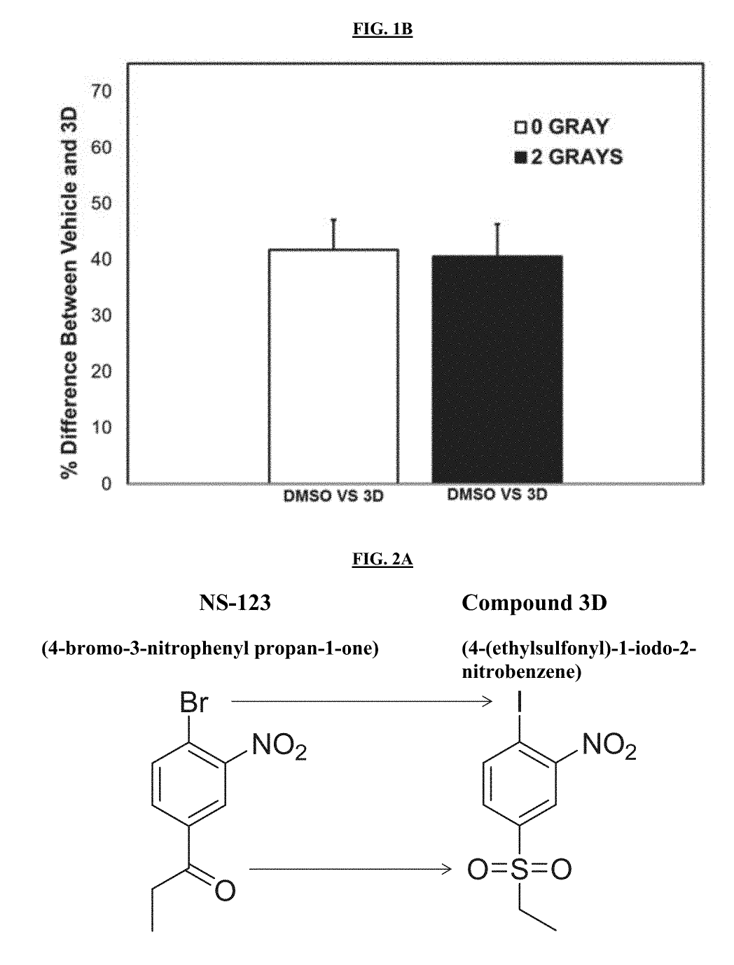

Compound 3D was previously shown to radiosensitize ER-positive breast cancer and prostate cancer cells (U.S. Pat. No. 8,541,476). This compound was then tested to see if it would radiosensitize aggressive TNBC cells such as MDA-MB-231. As shown in FIG. 1, while MDA-MB-231 cells responded favorably to a 2 Gray radiation dose by having significantly less colonies, compound 3D failed to enhance this action further: (reduction of 41.65%, 3D without radiation vs DMSO) and (reduction of 40.52%, compound 3D, 2 gray radiation vs DMSO), n=3, not statistically significant.

### EXAMPLE 2

### Compound 3D Kills MDA-MB-231 Cells by Itself Without the Need of Radiation, Through a Programmed Cell Death Pathway

As seen above, the lack of radiosensitizing capabilities of compound 3D was accompanied by an unusual basal effect of the compound without the radiation treatment on colony formation (reduction of 41.65%: 3D vs DMSO, FIG. 1).

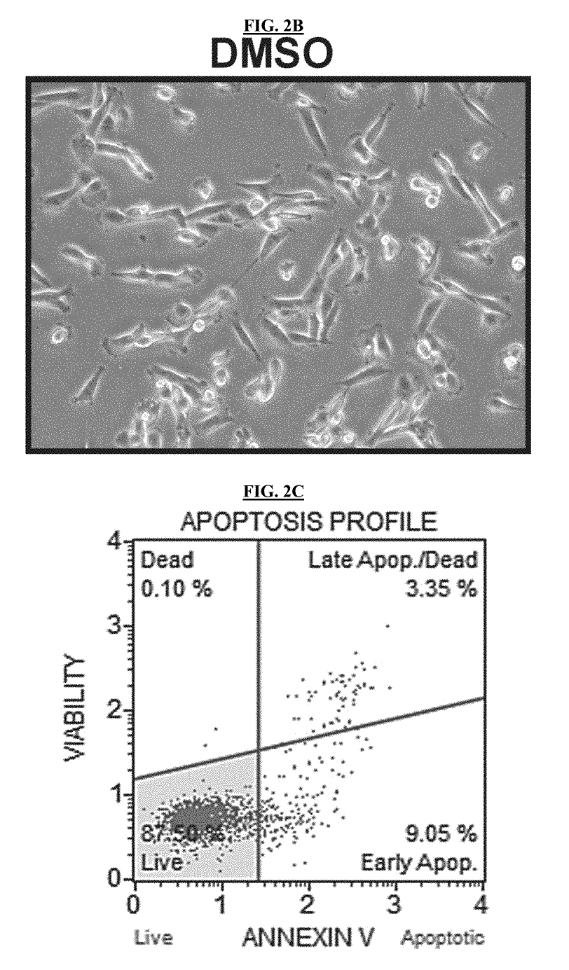

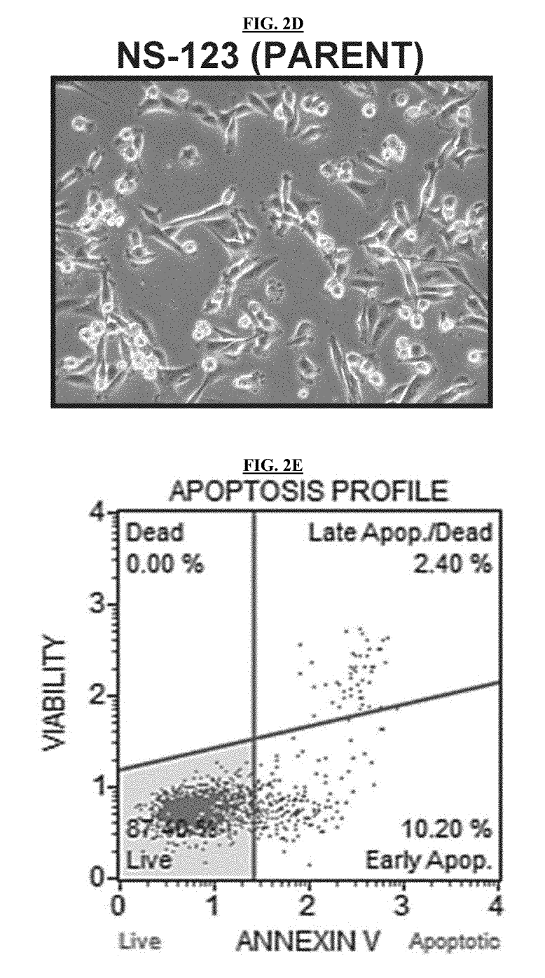

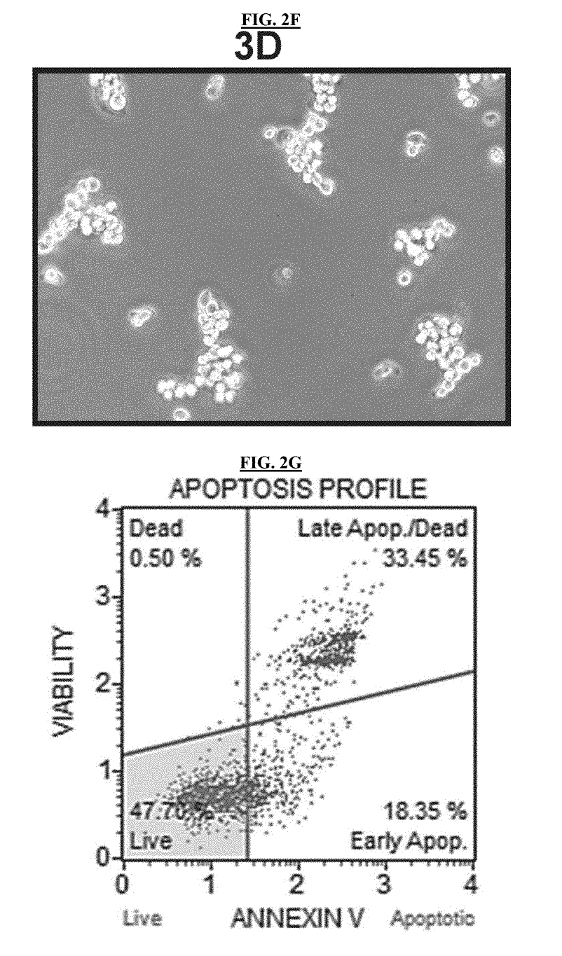

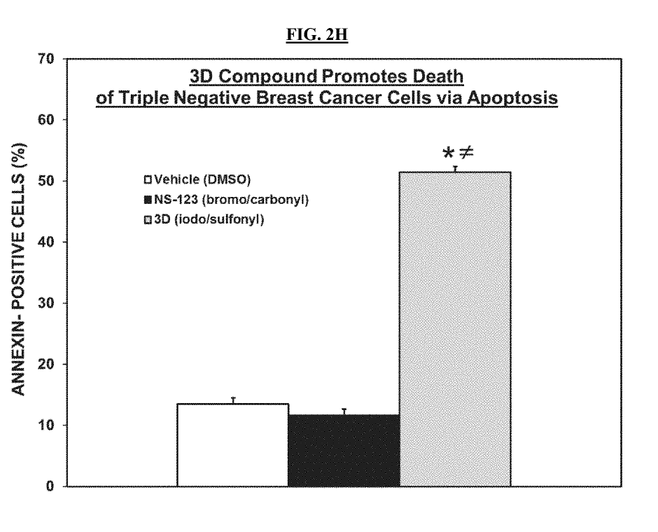

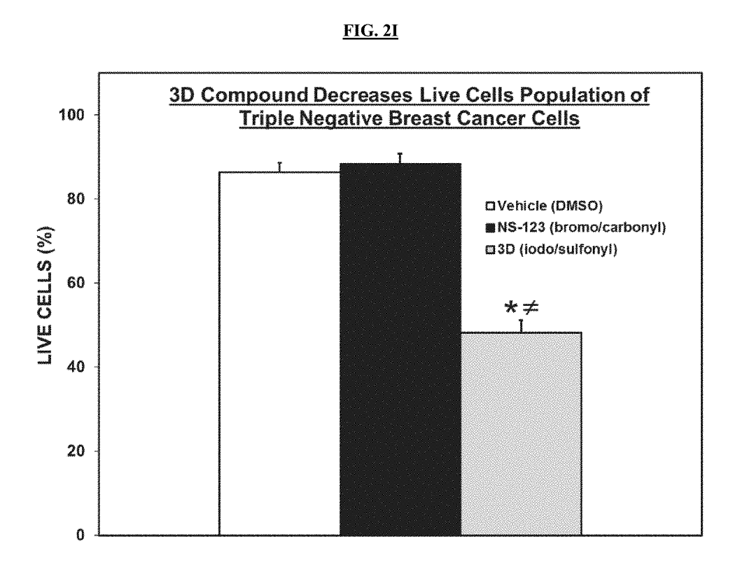

To further investigate the type of cell death induced by treatment of TNBC cells with 3D, its activity was compared to the original parent radiosensitizing compound NS-123 (4-bromo-3-nitrophenyl propan-1-one). FIG. 2A depicts the chemical structures of both the parent NS-123 (left) and compound 3D (right) highlighting the substitution with iodide and sulfonyl moieties in 3D structure compared to a bromide and carbonyl in the original radiosensitizing NS-123. As shown in FIG. 2F, MDA-MB-231 cells treated with compound 3D appeared rounded and unhealthy, while both DMSO (FIG. 2B) treated and radiosensitizer parent compound NS-123 (FIG. 2D) treated cells exhibited normal healthy features under a phase contrast microscope (10×).

To confirm what type of cell death was responsible for the observed activity, a quantitative annexin V death assay was performed to quantify the amount of necrosis (non-specific toxicity) vs apoptosis (programmed cell death) induced by compound 3D. Preliminary results suggested that the basal effects of compound 3D on TNBC cell were a calculated event rather than a non-specific necrotic occurrence. The apoptotic profiles in both DMSO (FIG. 2C) and NS-123 treated cells (FIG. 2E) displayed healthy populations (annexin V-negative: lower left quadrants), while compound 3D (FIG. 2G) pushed the cells to the upper right quadrant (annexin-V-positive), suggesting apoptosis mediated cell death. Compound 3D decreased live cell population from 86.4±2.2% (DMSO) to 48.1±3.0% (3D: p<0.001) and increased total annexin V-positive cells from 13.5±2.2% (DMSO) to 51.4±3.1% (3D: p<0.001). The parent NS-123 compound did not significantly change live cell population in MDA-MD-231 (88.3±2.4%, NS vs DMSO), nor did it induce any significant annexin V positivity (11.6±2.4%. NS vs DMSO), making compound 3D significantly different from NS-123 for both live and total apoptotic cells, p<0.001 (n=4-7 for all experiments).

### EXAMPLE 3

### Substitution of Para-Iodine (I) and/or Sulfone Leads to the Inhibition of Apoptotic Death in TNBC Cells, as Assessed by Caspase-3/7

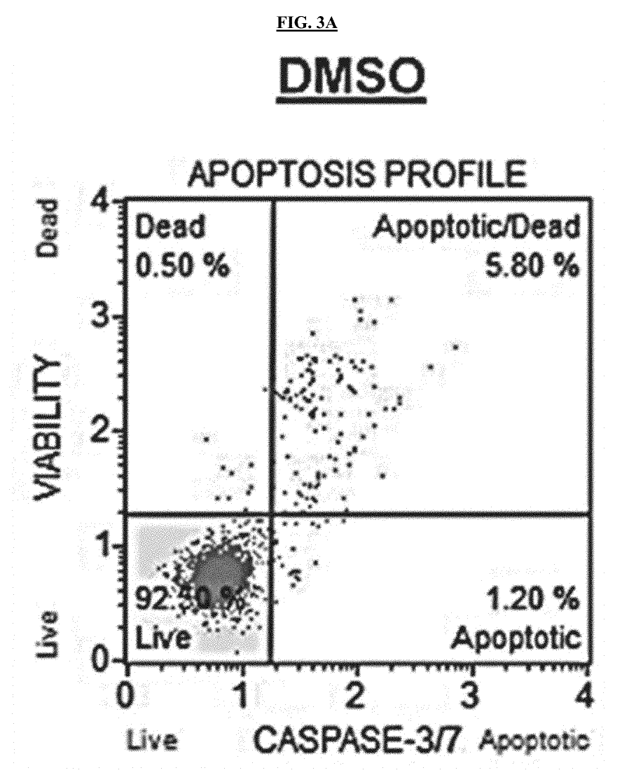

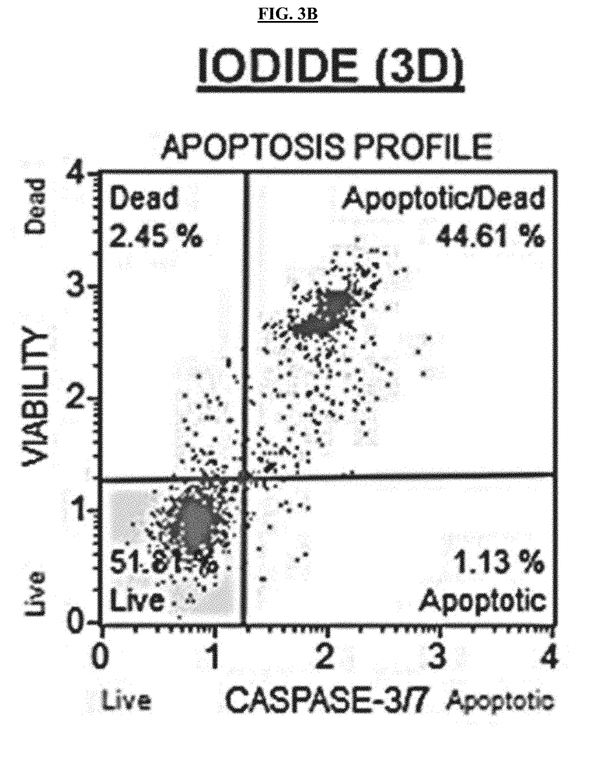

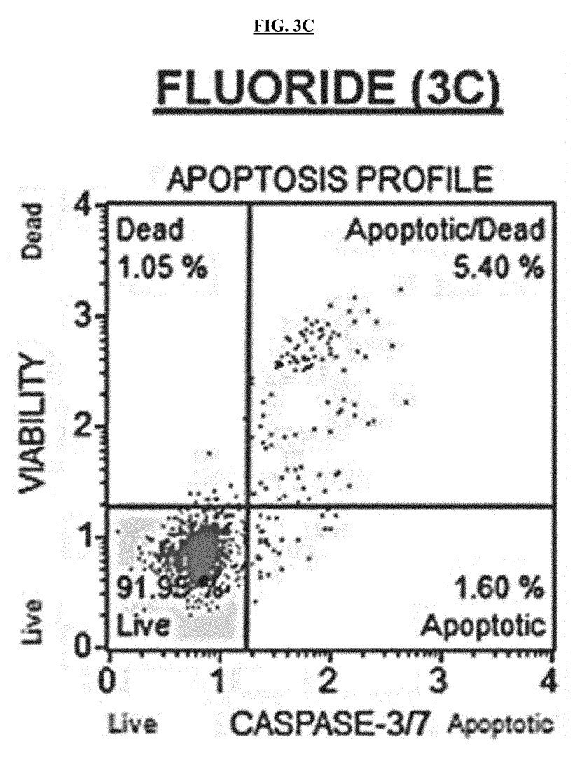

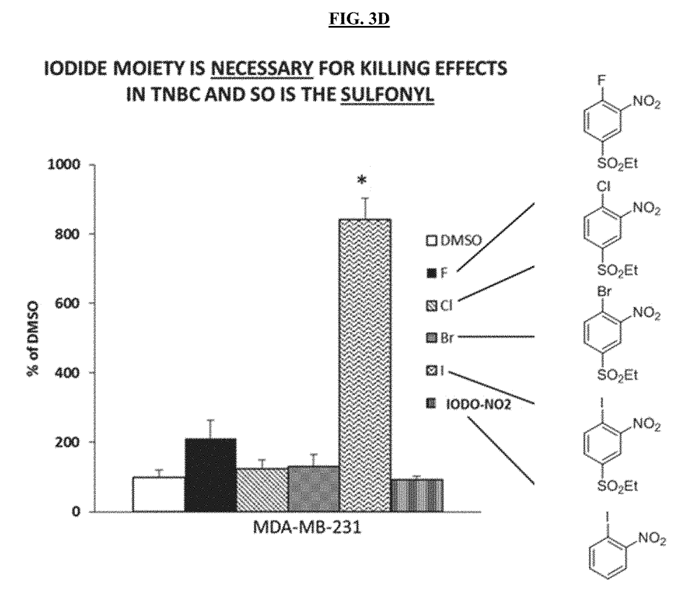

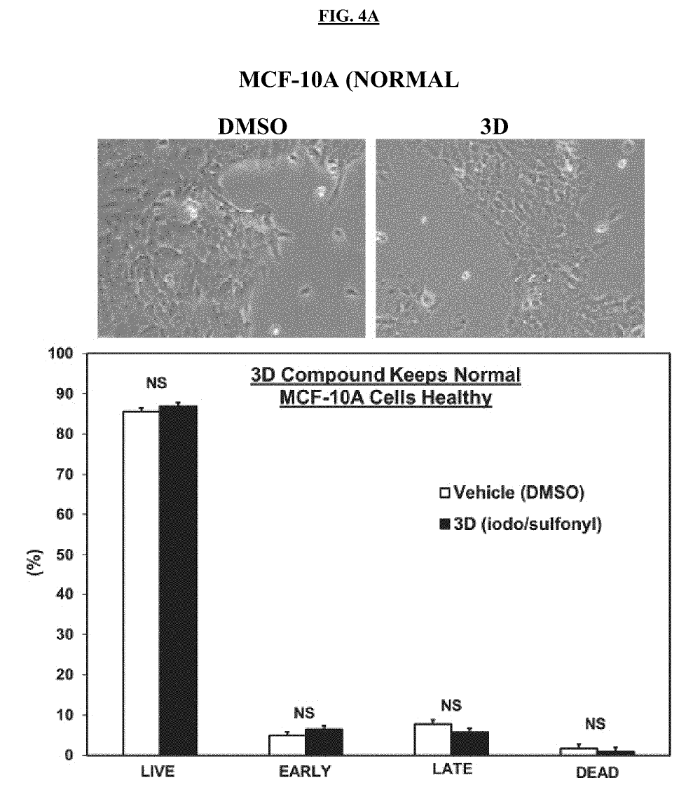

To identify mechanisms involved in the apoptotic death induced by compound 3D the iodine group (3D) was substituted by a fluorine group (3C) and a caspase 3/7 activity assay kit was utilized, which detects downstream effector apoptosis-causing caspases to assess death of TNBC cells. MDA-MB-231 cells treated with compound 3D induced specific caspase 3/7 activation. Compound 3D shifted the cell population to the upper right quadrant (caspase 3/7 active) while the DMSO control remained in the healthy lower left quadrant (caspase 3/7 inactive) (FIGS. 3A-3C). Quantitatively, 49.5±8.3% of cells were caspase 3/7 positive when treated with compound 3D compared to only 6.4±1.1% in the DMSO control group p<0.005 (FIG. 3D). The majority of these caspase 3/7-positive cells were associated with late apoptosis phase 47.4±8.5% vs DMSO control 5.8±1.0% (p<0.005), while the early apoptosis populations did not significantly differ, between 3D and DMSO control (0.9±0.2% vs 0.7±0.1%, respectively, p=NS), suggesting this compound acts extremely rapidly on downstream effector caspases in TNBC cells (-4 hrs).

The substitution of the iodine by a fluorine moiety at the para position caused a complete reversal of apoptosis (FIG. 3C). The levels of apoptosis as detected by caspases 3/7 positivity was completely reverted back to the levels observed in vehicle treated cells (7.9±3.0%). All experiments for caspase 3/7 activity were repeated on different passages (n=3). These results suggest that compound 3D caused the cells to reach late apoptosis in a short amount of time and that iodine plays an important part in killing TNBC cells.

Further substitution experiments were conducted, replacing the para iodine with chlorine or bromine, as well as experiments wherein the sulfone was removed. To help identify mechanisms involved in the apoptotic death induced by compound 3D, the iodine group was substituted with chlorine or bromine. A caspase 3/7 activity assay kit was used to detect downstream effector apoptosis-causing caspases to assess death of TNBC cells. MDA-MB-231 cells that were treated with compound 3D (iodine) induced specific caspase 3/7 activation, while all other halogen substitutions mentioned above failed to provide any significant killing abilities (FIG. 3D). Additionally, when a compound was generated lacking the sulfonyl group, all cytotoxic effects of 3D on TNBC cells was abolished. These results suggest that sulfonyl moiety contributes together with the iodine and nitro group to push TNBC cells towards cells death.

### EXAMPLE 4

### Compound 3D Shows Selectivity Towards TNBC Cells Killing While Sparing Normal Surrounding Cells

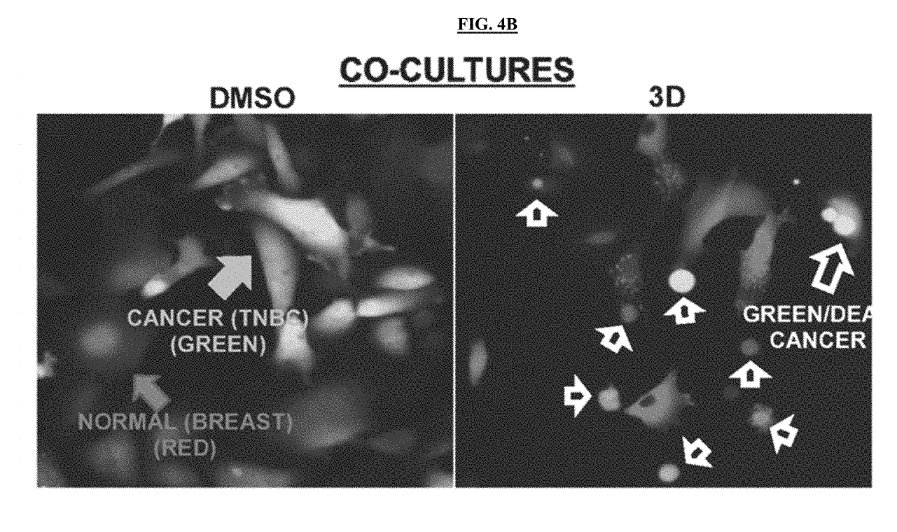

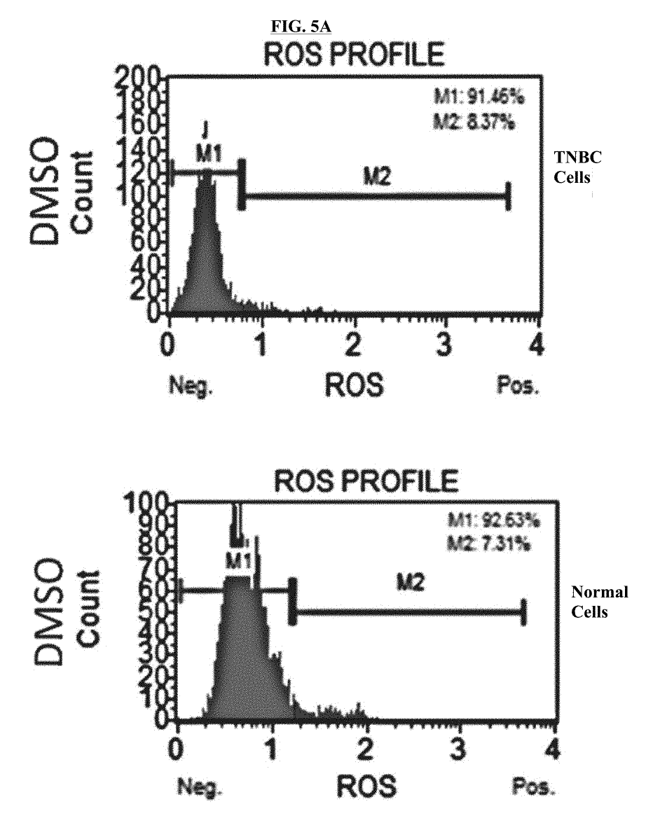

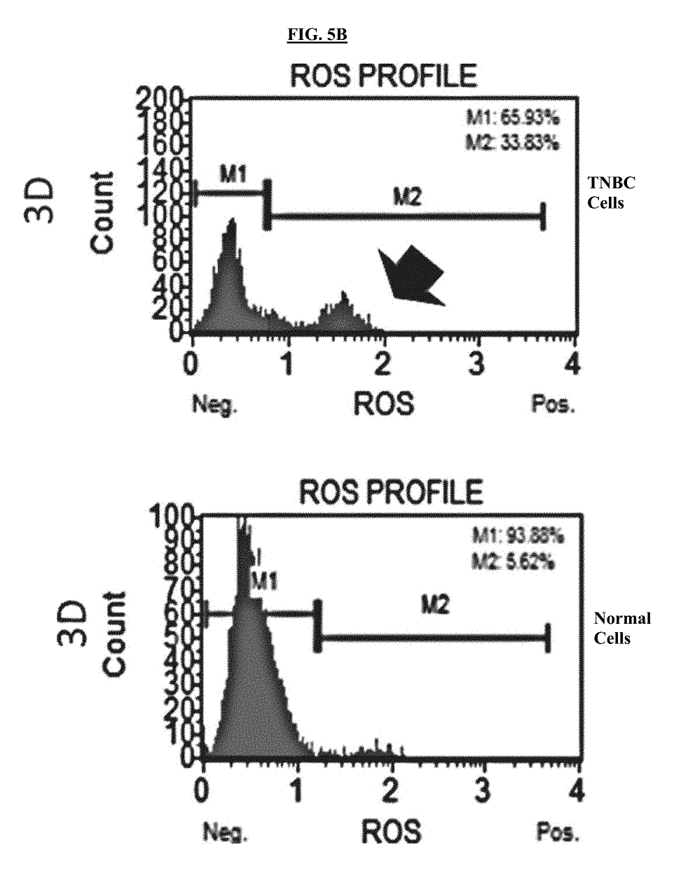

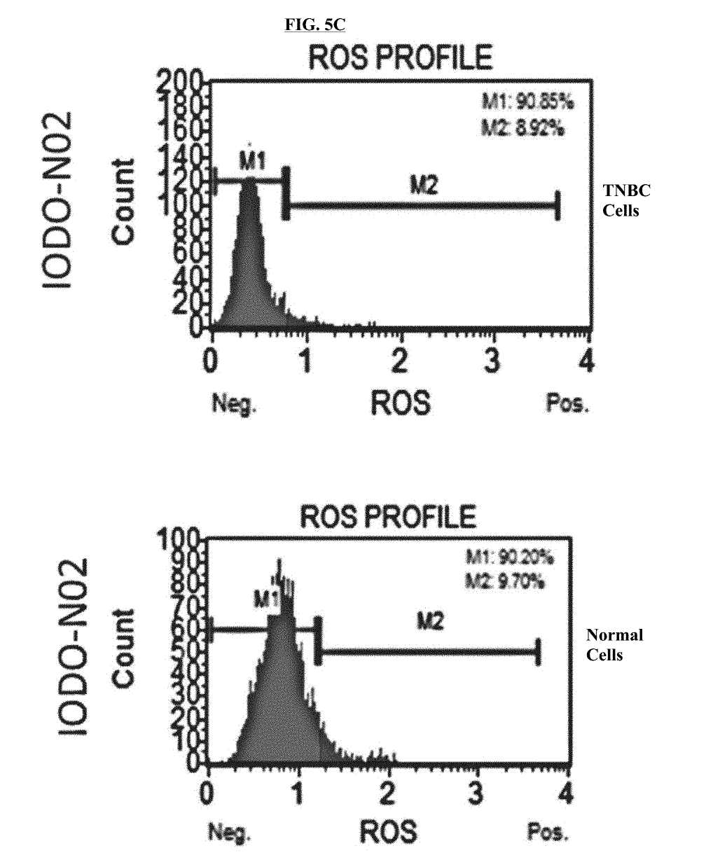

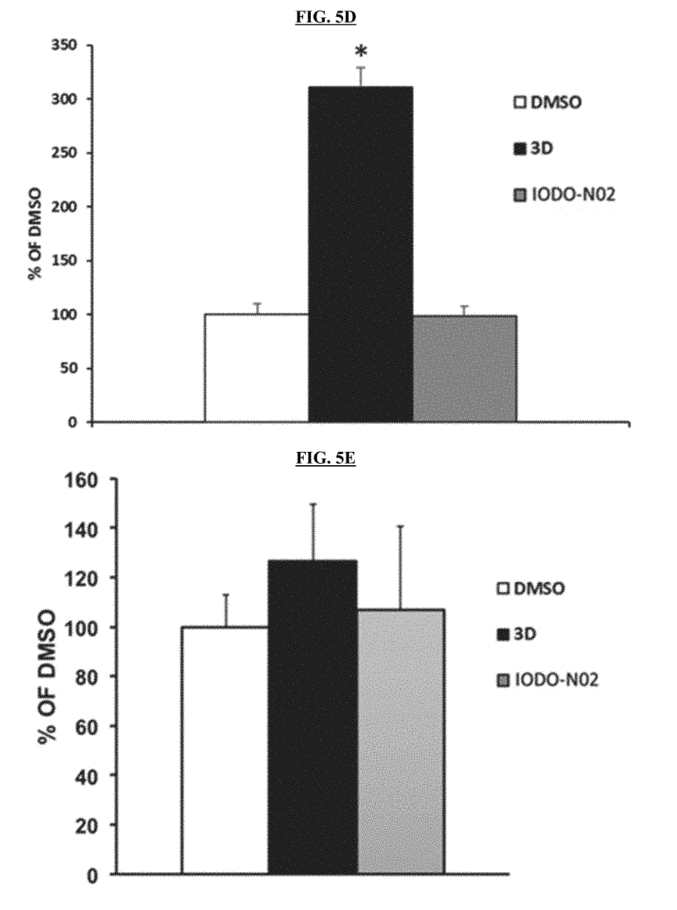

Since current clinical treatments for TNBC patients are indiscriminate and toxic to normal cells, new drug candidates which exhibit selectivity towards cancer cells while sparing normal surrounding cells are desired. The effects of compound 3D on survival of MCF-10A (normal mammary cells) were thus examined. As shown in FIG. 4A, pictures taken of normal MCF-10A cells following the treatment with compound 3D (at the same dose of 10 μM and same time point as shown in FIGS. 2F and 2G, demonstrate that the cells'morphology remains intact, suggesting that normal cells are protected against the killing effects of compound 3D. Using the cell death quantitative assay (Annexin V assay), 3D does not significantly alter cell viability in normal MCF-10A cells with 85.6±5.9% (DMSO) vs 86.9±3.3% (3D), p=NS and total apoptosis also did not significantly differ 12.7±5.3% (DMSO) vs 12.2±3.2% (3D), p=NS. All experiments were repeated on different passages (n=5).

To validate the selective nature of 3D towards cancer cells, normal human mammary cells were co-cultured (MCF-10A) with human TNBC cells (MDA-MB-231) in the same culture dish (FIG. 4B) and incubated with 3D to assess the compound's selectivity. This experiment was done by plating a 1:1 ratio of MCF-10A cells (mcherry red) and MDA-MB-231 cells (GFP-green) and challenging them with DMSO or 3D (10 μM) for 4 hrs as described in Example 2. Following the treatments, pictures were acquired at 20× using the EVOS-Fl microscope. As seen in FIG. 4B, when co-cultures were challenged with compound 3D, apoptotic death selectively occurred in the green cells (TNBC) while the red cells (normal) remained healthy. This co-culture was repeated 3 separate times on different passages (n=3). These results further suggest a selective preference of 3D for killing cancer cells while acting as a completely inert compound around normal non-cancerous cells.

### EXAMPLE 5

### Induction of Reactive Oxygen Species Formation by 3D in TNBC.

Manipulation of reactive oxygen species (ROS) levels is one potential mechanistic target for anti-cancer molecules because high levels of ROS can promote cell death. It was found that 3D significantly raised the levels of ROS in TNBC cells (MDA-MB-231 cells) while lacking any significant effects in normal breast cells (FIGS. 5A-5E). Levels of ROS were detected by assessing the conversion of a non-fluorescent compound (dihydroethidium) to its oxidized metabolite (ethidium bromide) in the presence of intracellularly accumulated ROS following 3D administration using a Muse oxidative stress kit.

3D demonstrated the ability to increase intracellular ROS levels in MDA-MB-231 triple negative cancer cells while leaving the ROS levels in normal breast cells unchanged. Without intending to be limited to any particular theory, it is possible that ROS neutralizing enzymes could be inhibited by 3D in TNBC cells, thus causing ROS levels to accumulate rapidly, killing the cells through apoptosis due to irreparable DNA damage. Typically, ROS neutralizing enzymes are elevated in cancer cells to prevent toxic ROS accumulation. Examples of these ROS scavenging enzymes are superoxide dismutase (SOD), catalase, glutathione peroxidase, thioredoxin and glutaredoxin. Alternatively, 3D could promote the activation of ROS generating systems. A differential ROS neutralizing or ROS producing capability within TNBCs and normal cells could explain the selective effects observed.

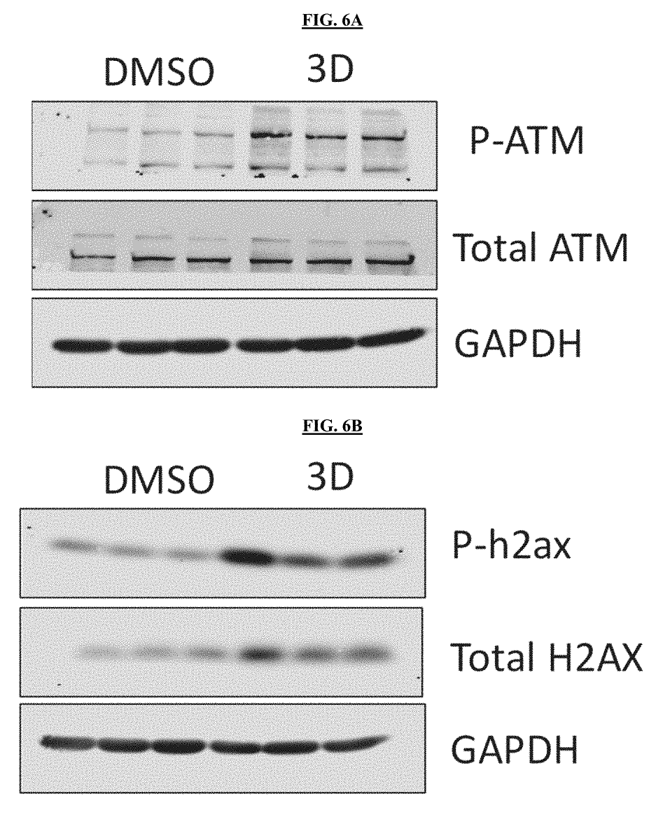

It was found that 3D caused a significant increase in phosphorylation (activation) of ATM and H2AX following 4 hours of treatment (FIGS. 6A-6B). H2AX and ATM are markers of DNA damage, thus suggesting the presence of DNA damage in cells after exposure to 3D. Without intending to be limited to any particular theory, together, these mechanistic results suggest that 3D might cause a rapid rise in ROS inside TNBC cells which causes irreparable DNA breaks leading to apoptotic cell death as described in Example 3 through the activation of caspase3/7.

### EXAMPLE 6

### Use of 3D to Treat Additional Triple Negative Cell Lines

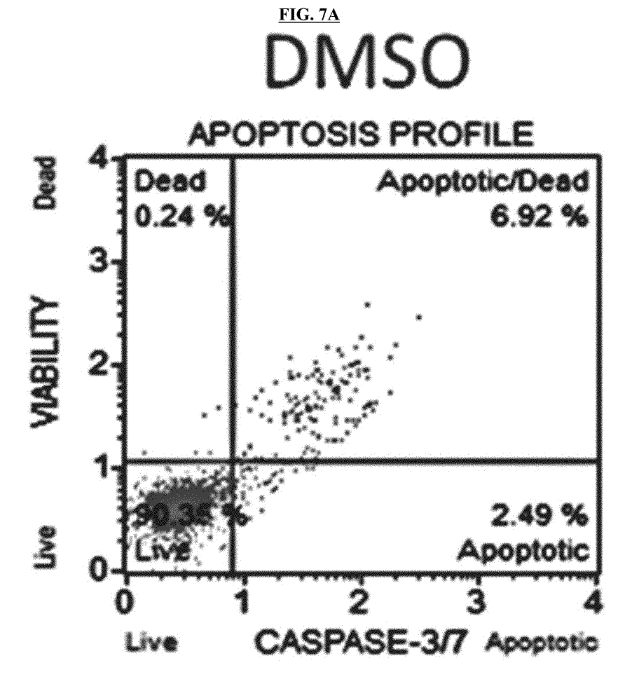

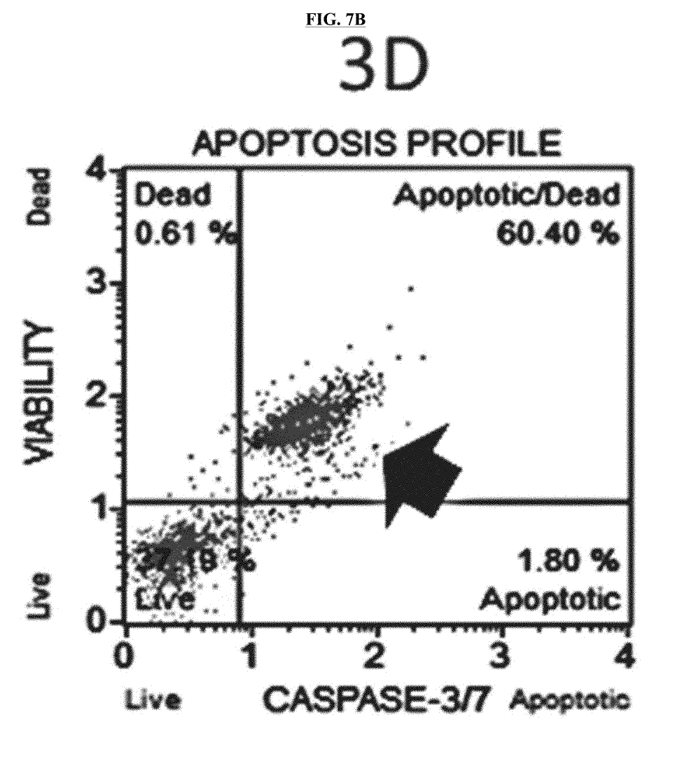

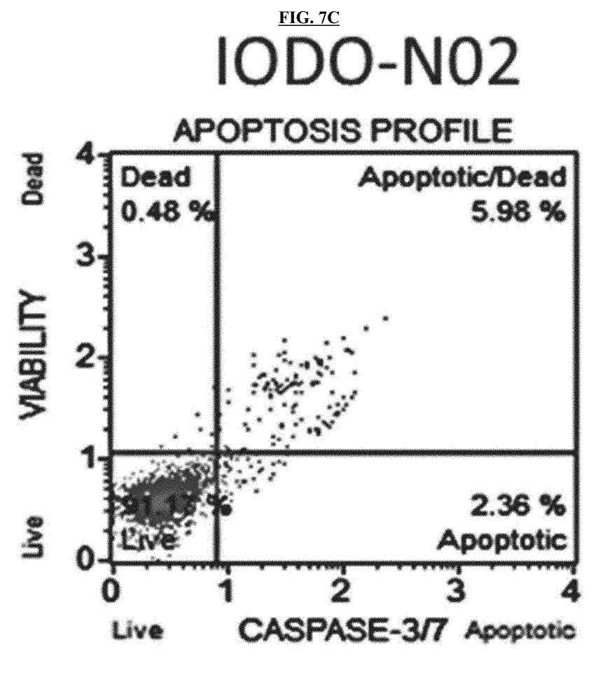

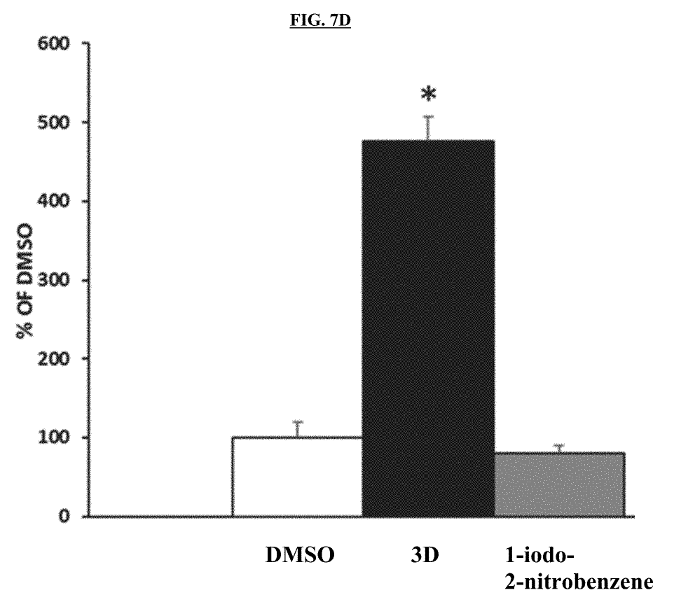

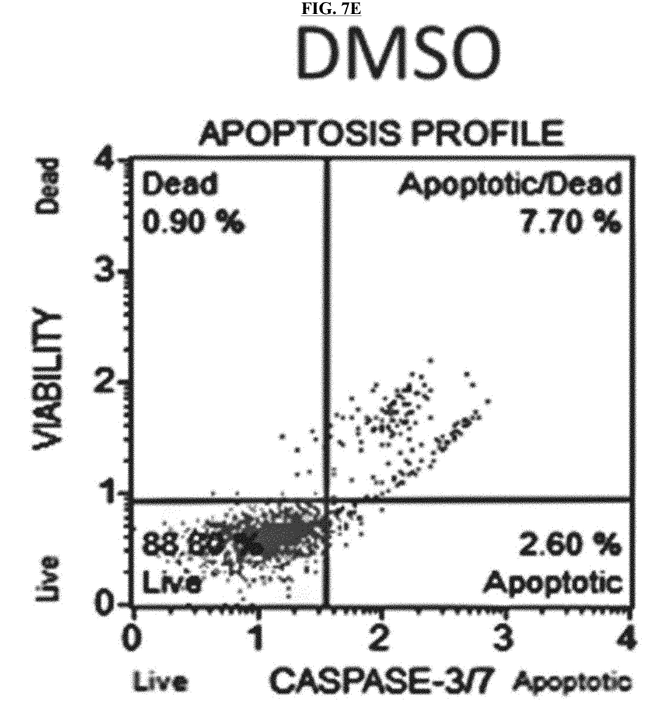

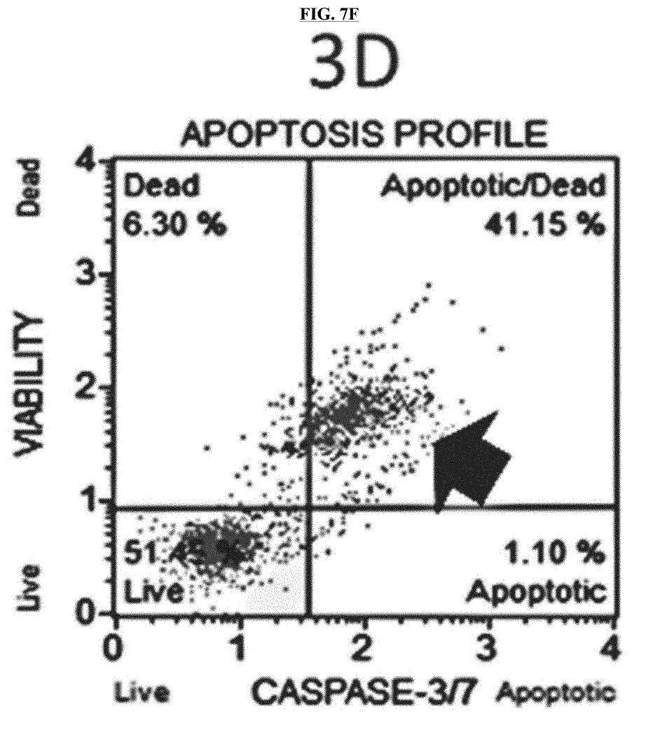

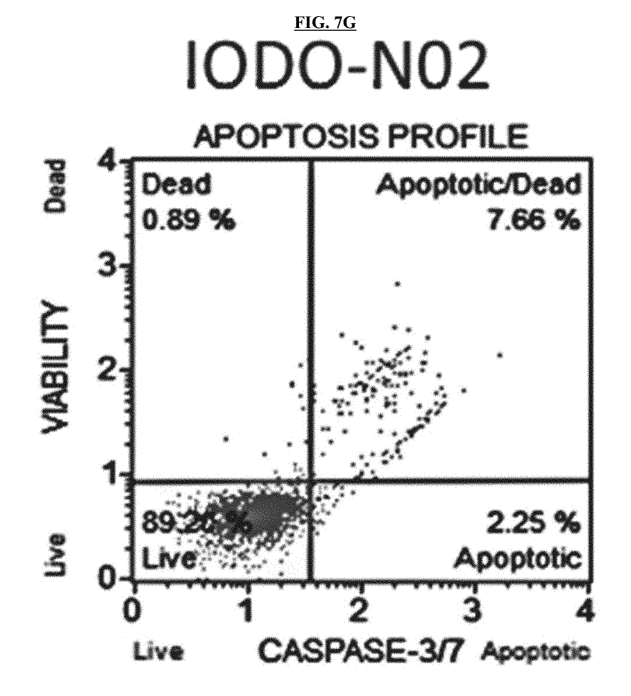

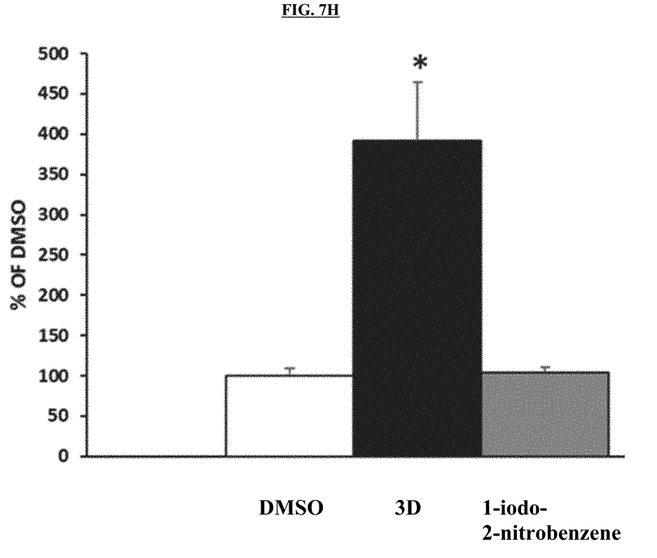

In order to explore the broader effects of 3D in the killing of TNBC cells, the experiments reported in Example 2 were repeated in 2 additional cells lines BT-549 and Hs578T. As observed for MDA-MB-231 cells, both BT-549 and HS578T demonstrated increased caspase 3/7 activation following 4 hr treatment with 3D (10 μM) (FIGS. 7A-7H).

The disclosures of each and every patent, patent application, and publication cited herein are hereby incorporated herein by reference in their entirety. While this invention has been disclosed with reference to specific embodiments, it is apparent that other embodiments and variations of this invention may be devised by others skilled in the art without departing from the true spirit and scope of the invention. The appended claims are intended to be construed to include all such embodiments and equivalent variations.

What is claimed is:

1. A method of treating or preventing triple negative breast cancer (TNBC) in a subject, the method comprising administering to the subject a therapeutically effective amount of the compound of formula (I), or a pharmaceutically acceptable salt or solvate thereof:

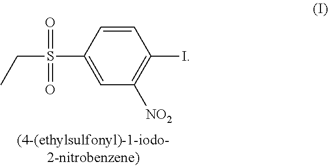

2. The method of claim 1, wherein the administration of the compound causes apoptosis in TNBC cells.

3. The method of claim 2, wherein the administration of the compound does not cause any, or causes insignificant, apoptosis in non-cancerous cells.

4. The method of claim 1, wherein the administration of the compound increases the concentration of reactive oxygen species in TNBC cells.

5. The method of claim 1, wherein the administration of the compound does not increase the concentration of reactive oxygen species in non-cancerous cells.

6. The method of claim 1, wherein the administration of the compound of formula (I) induces DNA damage in TNBC cells.

7. The method of claim 1, wherein the compound is administered as part of a pharmaceutical composition.

8. The method of claim 1, wherein the subject is not administered any additional chemotherapeutic agent or anti-cell proliferation agent.

9. The method of claim 8, wherein the subject is not administered any additional chemotherapeutic agent or anti-cell proliferation agent in an amount sufficient to treat or prevent TNBC in the subject.

10. The method of claim 1, wherein the subject is not subjected to ionizing radiation.

11. The method of claim 1, further comprising administering to the subject at least one additional agent selected from the group consisting of a chemotherapeutic agent, an anti-cell proliferation agent, a gene therapy agent, and a immunotherapy agent.

12. The method of claim 11, wherein the compound and the at least one additional agent are co-administered to the subject.

13. The method of claim 12, wherein the compound and the at least one additional agent are coformulated.

14. The method of claim 11, wherein the at least one additional agent is selected from the group consisting of taxotere, cyclophosphamide, paclitaxel, fluorouracil, doxorubicin, and cycloheximide.

15. The method of claim 1, wherein the subject is a mammal.

16. The method of claim 15, wherein the subject is a human.
